SEQUENCE LISTING (12 sequences)

SEQ ID NO: 1 - DNA, 22 bp, Artificial Sequence
  HOXA9 forward primer
gggttttaygaggygttaaata

SEQ ID NO: 2 - DNA, 20 bp, Artificial Sequence
  HOXA9  reverse primer
aaaaccctaaaccaacctcc

SEQ ID NO: 3 - DNA, 25 bp, Artificial Sequence
  HOXA9 FAM probe
acaaacgacgacgctaaccgaacac

SEQ ID NO: 4 - DNA, 21 bp, Artificial Sequence
  SCT forward primer
tgttggggtattaygttagga

SEQ ID NO: 5 - DNA, 22 bp, Artificial Sequence
  SCT reverse primer
aacrttcaccaacraactcaac

SEQ ID NO: 6 - DNA, 30 bp, Artificial Sequence
  SCT HEX probe
aaaaaacgcgcgactccaacgactactaca

SEQ ID NO: 7 - DNA, 20 bp, Artificial Sequence
  KCNS2 forward primer
yggtaagttttaygttatgg

SEQ ID NO: 8 - DNA, 30 bp, Artificial Sequence
  KCNS2 reverse primer
aactataactacaacaaaaatcaataaaaa

SEQ ID NO: 9 - DNA, 30 bp, Artificial Sequence
  KCNS2 FAM probe
tcgttaataccccaatactcgatctcctaa

SEQ ID NO: 10 - DNA, 23 bp, Artificial Sequence
  BARHL2 forward primer
gtattaattttygaatagggaga

SEQ ID NO: 11 - DNA, 22 bp, Artificial Sequence
  BARHL2 reverse primer
tccaataccaattcaaacaacc

SEQ ID NO: 12 - DNA, 30 bp, Artificial Sequence
  BARHL2 HEX probe
taataaataaaaatttccgcccgctcgata

(19) United States
(12) Patent Application Publication
(10) Pub. No.: US 2024/0068043 A1
(43) Pub. Date: Feb. 29, 2024

(54) METHOD AND KIT FOR MONITORING NON-SMALL CELL LUNG CANCER

(71) Applicant: NATIONAL TAIWAN UNIVERSITY, Taipei (TW)

(72) Inventors: Hsing-Chen TSAI, Taipei City (TW); Chong-Jen YU, Taipei City (TW); Hsuan-Hsuan LU, Taipei City (TW); Shu-Yung LIN, Taipei City (TW); Yi-Jhen HUANG, Taipei City (TW); Chen-Yuan DONG, Cupertino, CA (US)

(73) Assignee: NATIONAL TAIWAN UNIVERSITY, Taipei (TW)

(21) Appl. No.: 18/279,824

(22) PCT Filed: Mar. 1, 2022
(86) PCT No.: PCT/US2022/018340

§ 371 Date: Aug. 31, 2023

Related U.S. Application Data
(60) Provisional application No. 63/154,837, filed on Mar. 1, 2021.

(51) Int. Cl. C12Q 1/6886

(52) U.S. Cl. CPC C12Q 1/6886; C12Q 2600/112; C12Q 2600/118; C12Q 2600/154; C12Q 2600/16

(57) ABSTRACT

Provided is a method for diagnosing and monitoring progression of cancer or effectiveness of a therapeutic treatment. The method includes detecting a methylation level of at least one gene in a biological sample containing circulating free DNA. Also provided are primer pairs and probes for diagnosis or prognosis of cancer in a subject in need thereof.

### BACKGROUND

### Technical Field

The present disclosure relates to methods or kits for monitoring non-small cell lung cancer in a subject in need thereof. The present disclosure also relates to methods, compositions and kits for detecting, diagnosing, prognosing, and characterizing non-small cell lung cancer in a subject in need thereof.

### Description of Related Art

Cancers remain the leading causes of death worldwide. International Agency for Research on Cancer (IARC) estimated 10 million deaths and 19.3 million cases in 2020 in the recently released updates in Globocan 2020.

Among all cancers, worldwide incidences of lung cancer rank top three in both men and women. An enormous number of lung cancer patients are therefore under treatment and follow-ups, and require regular evaluation on their responses to treatment and on progression of the cancer.

Presently, diagnosis of cancer and evaluation of responses to various treatments primarily rely on imaging-based methods, such as computed tomography (CT) or nuclear scans that are subjected to variable sensitivities, or invasive tumor biopsy. However, both of these evaluation methods are hardly repeatable. For instance, detection of disease progression may be obscured by noncancerous parenchymal changes such as inflammation or fibrosis. Therefore, it takes significant changes in tumor sizes to confirm disease status alterations. In fact, few diagnostic modalities are available to track the dynamics of disease over the treatment course.

In addition, since the lung cancer patients need to be followed-up and evaluated regularly, minimally invasive methods that allow serial monitoring of patient's treatment responses and disease progression are highly desirable for both patients and medical physicians.

### SUMMARY

Herein, the present disclosure is therefore provided with at least one biomarker and a method to characterize, diagnose, prognosticate, stratify and monitor the progression or recurrence of non-small cell lung cancer in a subject in need thereof. The present disclosure provides a method and biomarker that detect disease progression in a subject preceding radiographic detection, regardless of the mutation status or ethnic group of the subject.

In at least one embodiment, a method is provided to characterize non-small cell lung cancer in a subject in need thereof, comprising detecting a methylation level of at least one gene selected from the group consisting of KCNS2, HOXA9, SCT, BARHL2, and any combination thereof in a biological sample from the subject, wherein the biological sample contains circulating free DNA.

In at least one embodiment, the method of the present disclosure further comprises detecting a methylation level of KCNS2. In some embodiments, the methylation level is detected by: at least one primer and a probe, wherein the primer pair has at least 80% sequence identity to SEQ ID NOs: 1 and 2, respectively, and at least one probe having at least 80% sequence identity to SEQ ID NO: 3; the primer pair has at least 80% sequence identity to SEQ ID NOs: 4 and 5, respectively, and at least one probe having at least 80% sequence identity to SEQ ID NO: 6; the primer pair has at least 80% sequence identity to SEQ ID NOs: 7 and 8, respectively, and at least one probe having at least 80% sequence identity to SEQ ID NO:9; the primer pair has at least 80% sequence identity to SEQ ID NOs: 10 and 11 and at least one probe having at least 80% sequence identity to SEQ ID NO: 12; or any combination thereof.

In at least one embodiment of the present disclosure, the methylation level is detected by bisulfite sequencing, array or bead hybridization, quantitative real-time PCR, methylation-sensitive endonuclease digestion followed by sequencing, PCR and sequencing, methylation-specific PCR and pyrosequencing.

In at least one embodiment of the present disclosure, the method to characterize non-small cell lung cancer in a subject in need thereof further comprises calculating a methylation risk score based on the methylation level of the at least one gene. In some embodiments, the methylation level of the at least one gene is given different weights for calculating the methylation risk score. In some embodiments, the calculation of the methylation risk score further comprises at least one of age, gender, active smoking status and former smoking status. In at least one embodiment of the present disclosure, a mathematical model is used on the four genes to calculate circulating methylation risk scores in the plasma cfDNA measured by methylation-specific droplet digital PCR. In at least one embodiment of the present disclosure, a mathematical model is used in calculating clinical methylation risk scores for lung cancer detection in the training and the validation cohorts of lung cancer patients at all stages. In at least one embodiment of the present disclosure, the methylation risk score is calculated as −1.547+0.024*SCT+0.084*HOXA9-0.039*B ARHL2+0.031*KCNS2.

In at least one embodiment of the present disclosure, the biological sample used in the method to characterize non-small cell lung cancer in a subject is a body fluid, e.g., blood, sputum, pleural fluid, cerebrospinal fluid or any combination thereof. In at least one embodiment of the present disclosure, the at least one gene tested in the method to characterize non-small cell lung cancer in a subject carries a driver mutation, a passenger mutation, or a combination thereof.

In the present disclosure, a kit for characterizing non-small cell lung cancer is also provided. In at least one embodiment, the kit comprises a primer pair and a probe for detecting a methylation level of at least one gene, wherein: the primer pair has at least 80% sequence identity to SEQ ID NOs: 1 and 2, respectively, and the probe has at least 80% sequence identity to SEQ ID NO: 3; the primer pair has at least 80% sequence identity to SEQ ID NOs: 4 and 5, respectively, and the probe has at least 80% sequence identity to SEQ ID NO: 6; the primer pair has at least 80% sequence identity to SEQ ID NOs: 7 and 8, respectively, and the probe has at least 80% sequence identity to SEQ ID NO: 9; or the primer pair has at least 80% sequence identity to SEQ ID NOs: 10 and 11, respectively, and the probe has at least 80% sequence identity to SEQ ID NO: 12; or any combination thereof.

In the present disclosure, a method of evaluating a therapy of non-small cell lung cancer is also provided. The method comprises obtaining a biological sample comprising circulating free DNA from a subject who had received the therapy of non-small cell lung cancer; detecting a methylation level of at least one gene selected from the group consisting of KCNS2, HOXA9, SCT, BARHL2, and any combination thereof; calculating a methylation risk score based on the methylation level of the at least one gene, wherein a change of the methylation risk score is indicative of efficacy of the therapy of non-small cell lung cancer.

In at least one embodiment of the present disclosure, the method further comprises detecting a methylation level of KCNS2. In some embodiments, an increase of the methylation risk score is indicative of disease progression. In at least one embodiment, the therapy of non-small cell lung cancer is surgery, radiation therapy, chemotherapy, targeted therapy, immunotherapy. or any combination thereof. In at least one embodiment, the disease progression comprises increase in tumor size or metastasis.

### BRIEF DESCRIPTION OF DRAWINGS

The present disclosure will become more readily appreciated by reference to the following descriptions in conjunction with the accompanying drawings.

FIG. 1 shows the flow chart of the procedures carried out in discovering biomarkers, developing assays with the biomarkers, and validating the biomarkers in the present disclosure. Marker discovery phase: In-silico analysis of genome-wide methylation data from surgically resected tumor tissues from the National Taiwan University Hospital and The Cancer Genome Atlas (TCGA) lung cancer cohorts. Differential methylation analyses between cancer tissues and adjacent lung tissues were performed to identify candidate methylated probes. Probes that were methylated in the peripheral blood mononuclear cells (PBMCs) of noncancer subjects from the Taiwan Biobank were excluded. A panel of cancer-specific methylated markers is designed for measurement by multiplex methylation-specific droplet digital PCR (MS-ddPCR), and these markers were verified using surgically resected lung cancer tissues. Assay development phase: The methylation levels of the identified markers in the circulating cell-free DNAs from lung cancer patients were quantified at initial diagnosis before treatment vs. noncancer subjects using MS-ddPCR. A model fitting with k-fold cross validation was performed to generate methylation risk scores based on the marker panel. Longitudinal validation phase: The cfDNA methylation scores of 268 samples (58 lung cancer patients) were obtained at initial diagnosis and follow-up visits every 3 months over the course of treatment. The relationships between methylation scores and radiological/clinical responses were recorded.

FIGS. 2A to 2C show discovery of markers from genome-wide methylation data of primary lung cancer tissues. FIG. 2A illustrates the heatmaps showing the methylation levels (β values) of the 4-probe panel (SCT, KCNS2, HOXA9 and BARHL2) measured by Infinium MethylationEPIC BeadChips and HumanMethylation450 in the surgically resected lung cancer and the adjacent lung tissues from the NTUH (Adeno (lung adenocarcinoma tissues): 30; SqCC (lung squamous cell carcinoma tissues): 35; AdjU (adjacent unaffected tissues): 15) and TCGA cohorts (Adeno 460, SqCC 370, AdjU 74). Methylation levels of these probes in the peripheral blood mononuclear cells (PBMCs) of noncancer subjects from the Taiwan Biobank (N=550) were used as controls. NTUH: National Taiwan University Hospital. TCGA: The Cancer Genome Atlas. β value: the ratio of the methylation signal to the sum of methylation and unmethylation signal for a given locus. FIG. 2B shows the β values of HOXA9, KCNS2, BARHL2 and SCT in lung cancer tissues vs. adjacent lung tissues (control, CTL) from the NTUH and TCGA cohorts measured by Infinium methylation arrays. FIG. 2C shows the summary of sensitivity, specificity, positive predictive value (PPV) and negative predictive value (NPV) based on different combinations of the 4-probe panel in lung adenocarcinoma and squamous cell carcinoma patients from the NTUH and TCGA cohorts. β values greater than 0.5 are considered positive for individual probes.

FIGS. 3A and 3B show methylation levels of classic methylated genes in the TCGA lung adenocarcinoma and squamous cell carcinoma tissues. FIG. 3A is the heatmap showing the β values of classic methylated genes in lung cancer measured by Infinium HumanMethylation450 BeadChips in the surgically resected lung cancer and adjacent lung tissues from the TCGA database (Adeno 460, SqCC 370, AdjU 74). FIG. 3B shows a summary of in silico sensitivity, specificity, positive predictive value (PPV) and negative predictive value (NPV) of the classic methylated genes in lung adenocarcinoma and squamous cell carcinoma patients from the TCGA database. The genes with the mean β value of promoter probes greater than 0.5 are considered methylated.

FIGS. 4A and 4B show the probe design and validation in primary lung cancer tissues. FIG. 4A shows the schematic diagrams of probe positions relative to individual marker genes, where positions and lengths of amplicons and CpG island are indicated. Arrows indicate the direction of transcriptions. FIG. 4B shows a heatmap of methylation levels of individual candidate probes for primary lung cancer tissues measured by methylation-specific real-time PCR. Lung cancer cell lines (i.e., A549, H1703, H1792 and H1299) were used as positive controls. The peripheral blood mononuclear cells (PBMCs) from healthy volunteers served as negative controls.

FIG. 5 shows the representative scatter dot plots of candidate methylated probes measured by methylation-specific droplet digital PCR (MS-ddPCR) in H1703 and A549 lung cancer cell lines. Experiments were performed in serial dilutions and compared between singleplex and multiplex PCRs. Each dot represented the PCR reaction signal in a droplet. The experimental condition for each lane was specified in the figure.

FIGS. 6A and 6B show the concentrations and fragment sizes of plasma cfDNA in non-small cell lung cancer (NSCLC) patients vs. noncancer control subjects. FIG. 6A shows the concentrations of plasma cfDNAs in NSCLC patients (N=74) and noncancer controls (N=63). ** p<0.01 by Welch's t test. FIG. 6B shows the fragment sizes of plasma cfDNAs in NSCLC patients (N=74) and noncancer controls (N=63). There are no significant (NS) differences in cfDNA fragment sizes between the two populations.

FIGS. 7A to 7D show that four-probe panel of methylated cfDNA markers in the plasma distinguishes between lung cancer patients and noncancer controls. FIG. 7A shows methylated signals (copies/mL) of individual gene probes (HOXA9, SCT, KCNS2, and BARHL2) in the plasma of non-small cell lung cancer patients (N=74) vs. healthy donors (N=63) measured by MS-ddPCR. * p<0.001 by Mann-Whitney U test. FIG. 7B shows the receiver operating characteristic (ROC) curve analysis of the four-probe panel and individual probes for the prediction of lung cancer versus noncancer controls. The area under the ROC curve (AUC) was 0.95 for the methylation scores calculated using the best model from the four-probe combination. FIG. 7C shows the percentages of clinically confirmed lung cancer patients who can be identified by methylation scores (>0.13) and carcinoembryonic antigen (CEA>5 ng/mL). FIG. 7D shows the relationships between methylation scores and smoking, gender, EGFR status, tumor staging, and metastatic sites. The central lines of box plots represent medians of methylation scores. The whiskers denote 1.5 * interquartile ranges (IQR). The p value was calculated by the Mann-Whitney test or one-way ANOVA with Tukey' s multiple comparison test. MPE: malignant pleural effusion. * p<0.05, ** p<0.01, *** p<0.001, **** p<0.0001. NS: not significant.

FIGS. 8A and 8B show the correlation plots of circulating methylated markers and the ages of subjects. FIG. 8A shows the scatter plots showing the correlation between the methylation levels of individual probes—HOXA9, SCT, KCNS2 and BARHL2 (y axis) and age (x axis). A linear regression line was fitted in each plot. The correlation coefficients (r) and p values were calculated by spearman correlation test. FIG. 8B shows the scatter plots showing the correlation between the methylation risk scores (y axis) and age (x axis). The methylation risk scores were calculated based on a weighted combination of the 4 probes.

FIGS. 9A to 9I show that methylation scores in serial blood samples correspond to clinical responses. FIG. 9A shows a swimmer plot of 58 lung cancer patients with serial blood samples (N=268). Each bar represents one subject in the study. Open bars represent patients with partial response or stable disease (PR/SD). Closed bars represent patients with progressive disease (PD). Open circles represent the timepoints of blood sampling. The stars mark the time of radiographic progression. PD: progressive disease. PR: partial response. SD: stable disease. FIG. 9B illustrates the spaghetti plots showing the dynamic changes in methylation risk scores during the treatment course for each lung cancer patient. Each line represents one subject in the study. Upper panel: patients with progressive disease. Lower panel: patients with partial response/stable disease. FIG. 9C shows the bar chart of estimated marginal means of methylation scores for patients with PD and those with PR/SD calculated from the generalized estimating equations (GEE) model. Error bars represent the 95% confidence interval (CI). FIG. 9D illustrates the bar chart showing methylation scores from individual visits of all patients during the treatment course categorized by different levels of methylation score (<0.13, 0.13-5.56, >5.56). The shade of the bar chart represents the disease status at the time of blood collection. FIG. 9E illustrates the dot plot showing the methylation levels of blood samples collected at the initial and follow-up visits. The disease status was defined according to the Response Evaluation Criteria in Solid Tumors (RECIST) criteria 1.1. PR, partial response; PD, disease progression; SD, stable disease. FIG. 9F shows the differences between the methylation risk score at each follow-up visit and that at initial visit (baseline). Increases in methylation scores are more associated with disease progression. The p value was calculated by the Mann-Whitney test. * p<0.05. FIG. 9G shows the differences between the methylation risk score at each follow-up visit and that at the prior visit. The p value was calculated by the Mann-Whitney test. *** p<0.001. FIG. 9H shows the methylation scores of each subject at initial blood collection categorized by PR/SD or PD. The p value was calculated by the Mann-Whitney test. FIG. 9I shows the Kaplan-Meier plot of progression-free survival between patients with low and high methylation scores (cutoff value=median) at baseline. The p value was calculated by the log-rank test.

FIGS. 10A to 10D show representative cases using the 4-gene methylation risk scores for disease monitoring. FIG. 10A shows a 46-year-old patient diagnosed with stage 4 lung adenocarcinoma carrying EGFR exon 19 deletions receiving erlotinib treatment. The patient achieved a partial response, and the disease was under control for approximately 800 days. Elevated methylation risk scores were noticed at the 10th and 11th visits. Enlargement of the primary lung tumor in the right upper lobe on chest tomography was observed at the 11th visit. FIG. 10B shows a 56-year-old patient diagnosed with stage 4 lung adenocarcinoma with bone metastasis. No specific driver mutations were identified. The patient received systemic chemotherapy with carboplatin and pemetrexed. The methylation score was decreased after the therapy but elevated at the next visit when there was no evidence of clinical and radiographic progression at the time. Three months later, chest tomography showed a significant progression of the primary lung tumor which was associated with a marked increase in the circulating methylation score. Inferior vena cava thrombosis was also noted. FIG. 10C shows a 69-year-old patient with stage 4 lung adenocarcinoma receiving erlotinib treatment. The patient's methylation scores decreased initially but increased again at approximately one year despite the shrinkage of the primary lung tumor on chest tomography. The methylation scores continued to rise until new bone metastases (bone mets) at the lumbar spine and pelvis were noted six months later. The methylation score declined rapidly after local palliative radiotherapy (RT). Conversely, there were no changes in the serum levels of carcinoembryonic antigen (CEA). FIG. 10D shows a 48-year-old patient with stage IIIb lung adenocarcinoma receiving concurrent chemoradiotherapy (CCRT) and gefitinib. Following CCRT, the patient's methylation scores declined and remained low for at least 600 days. The patient was clinically stable with no signs of tumor progression. The CEA levels were not informative throughout the treatment course.

FIG. 11 shows the flow chart of the procedures carried out in discovering biomarkers, developing assays with the biomarkers, and validating the biomarkers in the present disclosure. Marker discovery phase: In-silico analysis of genome-wide methylation data from surgically resected tumor tissues from the National Taiwan University Hospital and The Cancer Genome Atlas (TCGA) lung cancer cohorts. Differential methylation analyses between cancer and adjacent lung tissues were performed to identify candidate methylated probes. Probes that were methylated in the peripheral blood mononuclear cells (PBMCs) of non-cancer subjects from the Taiwan Biobank were excluded. A panel of cancer-specific methylated markers is designed for measurement by multiplex methylation-specific droplet digital PCR (MS-ddPCR), and these markers were verified using surgically resected lung cancer tissues. These lung tissues were LUAD: lung adenocarcinoma, LUSC: lung squamous cell carcinoma. Assay development phase: The methylation levels of the identified markers in the circulating cell-free DNAs from lung cancer patients were quantified at initial diagnosis before treatment vs. non-cancer subjects using MS-ddPCR. A logistical regression model was constructed to calculate methylation risk scores based on the four-gene combination in the training cohort. Validation phase: The cfDNA methylation scores were measured in the validation cohort (n=143) at initial diagnosis as diagnostic markers. For disease monitoring, a total of 261 samples (57 lung cancer patients) were obtained at initial diagnosis and follow-up visits every 3 months over the course of treatment. The relationships between methylation scores and radiological/clinical responses were recorded.

FIG. 12 shows the receiver operating characteristic (ROC) curve analysis of methylation risk scores and methylation levels of individual probes for the diagnosis of lung cancer in the training cohort of patients with stage IV lung adenocarcinoma, and the ROC curve analysis of methylation risk scores and methylation levels of individual probes for patients with stage IV lung cancer and for patients at all stages in the validation cohort.

FIG. 13 shows the relationships between methylation scores and clinical stages of non-small cell lung cancer. The values above the cutoff value (−0.764) are considered positive for the disease. The numbers and percentages of patients detected in each stage including in situ, stage I, II, III, and IV.

FIG. 14A shows a swimmer plot of 57 lung adenocarcinoma patients with serial blood samples (N=261). Each bar represents one subject in the study. Each bar represents one subject in the study. Bar color represents clinical responses (turquoise: PR/SD, red: PD). Yellow circles represent the timepoints of blood sampling. Yellow stars mark the time of radiographic/clinical progression. PD: progressive disease. PR: partial response. SD: stable disease. Gender, smoking history and EGFR status are denoted in the side bars on the left.

FIG. 14B shows a spaghetti plots demonstrating the dynamic changes in methylation risk scores during the treatment course for each lung cancer patient. Each line represents one subject in the study. Upper panel: patients with progressive disease (PD). Lower panel: patients with partial response/stable disease (PR+SD).

FIG. 14C shows a bar chart of estimated marginal means of methylation scores for patients with PD and those with PR/SD calculated from the generalized estimating equations (GEE) model. Error bars represent the 95% confidence interval.

FIGS. 15A to 15C show serial plasma cfDNA methylation scores for disease monitoring in three non-small cell lung cancer patients. FIG. 15A shows a serial plasma cfDNA methylation scores for disease monitoring in non-small cell lung cancer of patient L14, a 56-year-old patient diagnosed with stage 4 lung adenocarcinoma with bone metastasis. Patient L14's disease was initially under control by carboplatin and pemetrexed. At approximately 9 months, chest tomography of patient L14 showed a significant progression of the primary lung tumor associated with a marked increase in the circulating methylation score. Inferior vena cava thrombosis was also noted in patient L14. FIG. 15B shows a serial plasma cfDNA methylation scores for disease monitoring in non-small cell lung cancer of patient L12, a 72-year-old patient with stage 4 lung adenocarcinoma receiving afatinib treatment. The patient L12 responded to the therapy, but a metastatic liver tumor was noted at approximately 270 days despite the continued shrinkage of the primary lung tumor. FIG. 15C shows a serial plasma cfDNA methylation scores for disease monitoring in non-small cell lung cancer of patient L44, a 69-year-old patient with stage 4 lung adenocarcinoma receiving erlotinib treatment. The methylation scores of patient L44d decreased initially but increased again at approximately 9 months. New bone metastases at the lumbar spine and pelvis of patient L44 were noted six months later. Remarkably, the methylation score declined rapidly after local palliative radiotherapy (RT) were observed.

### DETAILED DESCRIPTIONS

The present disclosure provides a method and biomarkers to characterize, diagnose, stratify, prognosticate and monitor non-small cell lung cancer in a subject in need thereof by analyzing the levels of one or more biomarkers in a sample obtained from the subject. In at least one embodiment of the present disclosure, the one or more biomarkers is a methylation level of at least one gene selected from the group consisting of HOXA9, KCNS2, SCT, and BARHL2. In at least one embodiment of the present disclosure, the methylation level of the at least one gene is measured in a biological specimen containing circulating tumor DNA or cell-free DNA.

In this disclosure, the methylation level of at least one gene is evaluated in a biological specimen containing cell-free DNA to characterize, diagnose, stratify, prognosticate and monitor non-small cell lung cancer in a subject in need thereof. In at least one embodiment, a methylation risk score is calculated based on the methylation level of one, two, three or four genes selected from the group consisting of HOXA9, KCNS2, SCT, and BARHL2. In at least one embodiment, the present disclosure provides at least one biomarker and a method in distinguishing between a lung cancer patient and a noncancer control. In at least one embodiment, the present disclosure provides a method for serial assessments of disease status and treatment responses.

Circulating free DNA (cfDNA) are DNA fragments released to the blood plasma. cfDNA can be used to describe various forms of DNA freely circulating in the bloodstream, including circulating tumor DNA (ctDNA), circulating cell-free mitochondrial DNA (ccf mtDNA), and cell-free fetal DNA (cffDNA). Circulating tumor DNA (ctDNA) originates from tumor cells through mechanisms such as apoptosis, necrosis, or active release, and can reflect molecular characteristics of original tumor tissues. In this disclosure, cfDNA and ctDNA may be used interchangeably to indicate the DNA fragments obtained from a biological specimen from a subject, such as blood plasma or other body fluid containing cfDNA. “Body fluid,” as used herein, is meant to be a biological sample obtained from a subject that is substantially devoid of intact cells. This may be derived from a biological sample that is itself substantially devoid of cells, or may be derived from a sample from which cells have been removed. Examples of cell-free body fluid include those derived from blood, such as serum or plasma; urine; or samples derived from other sources, such as semen, sputum, feces, ductal exudate, lymph, pleural fluid, cerebrospinal fluid or recovered lavage. cfDNA or ctDNA is used as a biological specimen containing a biomarker for monitoring therapeutic responses in cancer patients. Currently, most ctDNA assays are based on driver mutations such as EGFR and KRAS.

A driver mutation is a mutation that gives a selective advantage to a clone in its microenvironment through increasing either survival or reproduction thereof and therefore is causally implicated in oncogenesis. A driver mutation needs not be required for maintenance of the final cancer (although it often is), but it must have been selected at some points along the course of cancer development. A passenger mutation, on the other hand, has not been selected, has not conferred clonal growth advantage and has therefore not contributed to cancer development. Passenger mutations are found within cancer genomes because somatic mutations without functional consequences often occur during cell division. Thus, a cell that acquires a driver mutation will have biologically inert somatic mutations within its genome. These will be carried along in the clonal expansion that follows, and therefore will be present in all cells of the final cancer.

The number of driver mutations, and hence the number of abnormal cancer genes, in an individual cancer is not well established. It is highly likely that most cancers carry more than one driver mutations and that the number varies between cancer types. On the basis of age-incidence statistics, it has been suggested that common adult epithelial cancers such as breast, colorectal and prostate require 5 to 7 rate-limiting events, possibly equating to the number of driver mutations, whereas cancers of the hematological system may require fewer (Miller DG, “On the nature of susceptibility to cancer. The presidential address.” Cancer. 1980 Sep 15; 46(6): 1307-18).

Therefore, monitoring a disease status or treatment responses by testing for a specific driver mutation such as EGFR is limited to only a subset of patients. Furthermore, for each driver mutation, there exist dozens or hundreds of hotspots with dynamic clonal evolution occurring along the treatment course that may or may not correlate with clinical disease progression. In fact, the clonal evolution of cancer mutations is a frequent event during the treatment course. It requires a large panel of markers for better coverage, which is not only costly but also unsuitable for longitudinal monitoring of disease burden, while the targets for detection are continually changing. Unlike mutations that tend to have dozens to hundreds of hotspots, DNA methylation often takes place at a common region near the gene promoter and is better suited for longitudinal tracking of dynamic disease burdens.

DNA methylation is a biological process by which methyl groups are added to the DNA molecule. DNA methylation is one of many mechanisms in regulating a gene expression and can change the activity of a gene without changing the DNA sequence. When located in a gene promoter, DNA methylation typically acts to repress gene transcription, namely, to reduce gene expression and thereby suppress gene function. Aberrant DNA methylation is a hallmark of the carcinogenic process, as revealed by large-scale genomic/epigenomics studies. Similar to genetic mutations, DNA methylation is heritable and stable, thereby having great potential as a molecular marker. Unlike driver mutations with numerous hot spots, methylation regions around the promoter appear to be common for each gene across patient samples. Use of methylated ctDNA markers for longitudinal disease monitoring in lung cancer with or without driver mutations has yet to be explored.

In this disclosure, all terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

The term “to characterize” in a subject or individual may include, but is not limited to, to provide the diagnosis of a disease or a condition, to determine the stratification of a disease risk, to assess the risk of a disease or a condition, to provide the prognosis of a disease or a condition, to determine a disease stage or a condition stage, to determine the severity of a disease or a condition, to evaluate the malignancy potential of a disease or a condition, to monitor a recurrence of cancer, to evaluate a drug efficacy, to describe a physiological condition, to evaluate an organ distress or organ rejection, to monitor disease or condition progression, to determine therapy-related association to a disease or a condition, or to describe a physiological or biological state.

As used herein, prognosis of cancer may include predicting the clinical outcome of the patient, assessing the risk of cancer recurrence, determining treatment modality, or determining treatment efficacy.

As used herein, the term “metastasis” describes the spread of a cancer from one part of the body to another. A tumor formed by cells that have spread can be called a “metastatic tumor” or a “metastasis.” The metastatic tumor often contains cells that are similar to those in the original (primary) tumor, and have, but are not limited to, genomic, epigenetic, transcriptomic, and metabolic alterations.

As used herein, the term “progression” describes the course of a disease or a condition, such as a cancer, as it becomes worse or spreads in the body.

The terms “subject,” “patient” and “individual” are used interchangeably herein and refer to a warm-blooded animal, such as a mammal that is afflicted with, or suspected of having, at risk for or being pre-disposed to, or being screened for cancer, e.g., actual or suspected cancer. These terms include, but are not limited to, domestic animals, sports animals, primates and humans. For example, the terms refer to a human.

The term “detect,” “detecting” or “detection” includes assaying, or otherwise evaluating the target biomarker(s), such as the expression level or methylation level of selected gene(s) and the like, for ascertaining, establishing, characterizing, predicting or otherwise determining one or more factual characteristics of a cancer, such as stage, aggressiveness, metastatic potential or patient survival. A cut-off value or a standard may correspond to levels quantitated for samples from control healthy subjects with no disease or low-grade cancer or from other samples of the subject.

As used herein, the term “marker” or “biomarker” is a biological molecule, or a panel of biological molecules, whose certain characteristics such as methylation level or expression level in a tissue, cell or sample as compared to its level in normal or healthy tissue, cell or sample is associated with a disease state, such as an advanced stage of cancer progression, including disease in an early stage, e.g., prior to the detection of one or more symptoms associated with the disease.

As used herein, the term “sequence identity” or, for example, comprising a “sequence having 80% sequence identity to,” as used herein, refers to the extent that sequences are identical on a nucleotide-by-nucleotide basis over a window of comparison. Thus, a “percentage of sequence identity” may be calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence homology. Included in this disclosures are nucleotides having at least about 80%, at least about 83%, at least about 85%, at least about 88%, at least about 90%, at least about 92%, at least about 95%, at least about 97%, at least about 98%, at least about 99% or 100% sequence identity to any of the reference sequences described herein (see, e.g., Sequence Listing), typically where the nucleotide variant maintains at least one biological activity or function of the reference nucleotide, such as, maintaining their complementarity with the target sequence that are complementary to the reference nucleotide.

### EXAMPLES

Exemplary embodiments of the present disclosure are further described in the following examples, which should not be construed to limit the scope of the present disclosure.

Generally, the nomenclature used herein and the laboratory procedures utilized in the present disclosure include molecular, biochemical and recombinant DNA techniques. Such techniques are thoroughly explained in the literature. See, for example, “Molecular Cloning: A laboratory Manual,” Sambrook et al., (1989); “Current Protocols in Molecular Biology,” Volumes I-III Ausubel, R. M., ed. (1994); “A Practical Guide to Molecular Cloning,” John Wiley & Sons, New York (1988); Watson et al., “Recombinant DNA,” Scientific American Books, New York; Birren et al. (eds) “Cell Biology: A Laboratory Handbook,” Volumes I-III Cellis, J. E., ed. (1994); “Culture of Animal Cells—A Manual of Basic Technique,” Freshney, Wiley-Liss, N.Y. (1994), Third Edition; “Transcription and Translation,” Hames, B. D., and Higgins S. J., eds. (1984); “Animal Cell Culture,” Freshney, R. I., ed. (1986); “Immobilized Cells and Enzymes,” IRL Press, (1986); “A Practical Guide to Molecular Cloning,” Perbal, B., (1984) and “Methods in Enzymology,” Vol. 1-317, Academic Press; “PCR Protocols: A Guide to Methods and Applications,” Academic Press, San Diego, Calif. (1990); Marshak et al., “Strategies for Protein Purification and Characterization—A Laboratory Course Manual,” CSHL Press (1996); all of which are incorporated by reference as if fully set forth herein. Other general references are provided throughout this disclosure. The procedures therein are believed to be well known in the art and are provided for the convenience of the readers. All the information contained therein is incorporated herein by reference.

### Genome-wide DNA Methylation Measurement of Surgically Resected Lung Cancer Tissues

Seventy-one surgically resected lung tumor tissues (lung adenocarcinoma (LUAD), N=39; lung squamous cell carcinoma (LUSC), N=32) and/or the adjacent unaffected tissues (N=17) from patients with non-small cell lung cancer were collected at the National Taiwan University Hospital (NTUH cohort) from Sep. 1994 to Oct. 2011. Informed consent was obtained prior to study enrollment for each patient. DNA extraction of primary tissues was performed using the traditional phenol-chloroform method. The extracted DNA was subjected to bisulfite treatment using the EZ DNA Methylation Kit (Zymo Research, Irvine, CA), followed by genome-wide methylation measurement using the Infinium MethylationEPIC Arrays (Illumina, San Diego, CA). Raw intensity data were obtained as IDAT files and processed using the R package “minfi” v1.30.01. Low-quality probes with a detection p-value>0.01 and probes described as single nucleotide polymorphisms (SNPs), cross-reactive and genetic variants were removed. The methylation data were uploaded to the Gene Expression Omnibus (GEO) with the accession number GSE159350. The study was approved by the Institutional Review Board (IRB) of NTUH (201701010RINB, 201605088RIND and 201809080RINC).

### Identification of Candidate Methylation Markers in Silico

The genome-wide methylation data of lung adenocarcinoma and squamous cell carcinoma from NTUH (NTUH cohort) and from The Cancer Genome Atlas (TCGA cohort) were used for marker identification. The Infinium Human Methylation 450K BeadChip data of the TCGA lung cancer cohorts were downloaded from the TCGA portal. As a control for tumor-derived DNA in the peripheral blood, the Infinium MethylationEPIC methylation data from the peripheral blood mononuclear cells (PBMCs) of 550 noncancer subjects from Taiwan Biobank were also obtained. Candidate cancer-specific methylated probes located within 1,500 bp around the transcription start sites were identified by differential methylation analyses between cancer tissues and unaffected adjacent tissues. Of all the identified probes, those that showed methylation signals in the peripheral blood mononuclear cells (PBMCs) of 550 noncancer subjects from the Taiwan Biobank were excluded. All data were analyzed using the R and Bioconductor packages.

### Enrollment of Lung Cancer Patients for Cell-free Tumor DNA Analysis

Patients over 20 years old who were newly diagnosed with stage III or IV non-small cell lung carcinoma at the National Taiwan University Hospital (NTUH) from Dec. 2015 to Nov. 2019 were prospectively enrolled. Pregnant or lactating women were excluded. For each participant, 10 mL of peripheral blood was collected before treatment and every three months until disease progression (enlarged primary tumor or presence of new metastasis) or death. Demographic data and clinical information including age, gender, smoking status, clinical stages, histological diagnosis, metastatic sites, mutation status and treatment regimens were recorded. For longitudinal follow-up of each patient, the treatment responses were evaluated based on RECIST criteria 1.1 (Eisenhauer E A, Therasse P, Bogaerts J, et al. New response evaluation criteria in solid tumors: revised RECIST guideline (version 1.1). Eur J Cancer. 2009;45(2):228-247). Medical records and images of each patient were independently reviewed by two pulmonologists to determine the status of disease progression (PD), partial response (PR) or stable disease (SD) at each follow-up visit during the treatment course until disease progression, change of treatment regimen or dropout of the study. Progression-free survival (PFS) was defined as the interval from study enrollment to the occurrence of PD. In addition, plasma cell-free DNAs (cfDNA) from healthy volunteers over 20 years old were collected as noncancer controls. The study was approved by the Institutional Review Board (IRB) of NTUH. Informed consent was obtained prior to the study enrollment for each patient (IRB#201510099RINC).

Isolation of cfDNA from Peripheral Blood Samples

The blood samples were collected in BD Vacutainer blood collection tubes containing K2EDTA (BD Biosciences, San Jose, CA) and immediately processed to obtain plasma by centrifugation at 1,000×g for 10 minutes at 4° C. (Eppendorf, Enfield, CT). For each patient or healthy volunteer, cfDNA extraction from 1 mL plasma was performed using the MagMAX Cell-Free DNA Isolation Kit according to the manufacturer's instructions (Thermo Fisher Scientific, Waltham, MA). Subsequently, the extracted cfDNA underwent bisulfite conversion using the EZ DNA Methylation Kit (Zymo Research, Irvine, CA). Quality control of cfDNA was performed by an Agilent 2100 Bioanalyzer using an Agilent High Sensitivity DNA Kit (Agilent, Santa Clara, CA) to check the distribution of fragment sizes. The cfDNA concentrations were measured by Qubit 2.0 Fluorometer using Qubit dsDNA HS Assay Kits (Thermo Fisher Scientific, Waltham, MA).

### Methylation-specific Droplet Digital PCR (MS-ddPCR)

Primers and probes specific for the identified genes including HOXA9, SCT, KCNS2 and BARHL2 were designed using MethPrimer 2.0 and shown in Table 1. PCR conditions were optimized using the genomic DNA of the H1703 lung cancer cell line (ATCC). The extracted plasma cfDNAs from lung cancer patients or noncancer controls were subjected to bisulfite conversion using an EZ DNA Methylation Kit (Zymo Research, Irvine, CA). Subsequently, the bisulfite-converted DNAs were added into the mixture of primers, probes and 2X supermix (Bio-Rad, Hercules, CA) with a final volume of 20 μL. The mixtures and droplet generation oil (Bio-Rad, Hercules, CA) were added into the DG8 Cartridge (Bio-Rad, Hercules, CA), and the QX200 Droplet Generator (Bio-Rad, Hercules, CA) was used to generate emulsions. The droplets were transferred to another 96-well plate (Bio-Rad, Hercules, CA) and placed in a standard PCR thermocycler after proper sealing. The cycling conditions were as follows: initial degeneration at 95° C. for 10 minutes, followed by 40 cycles of denaturation at 94° C. for 30 seconds and annealing at 55° C. for 60 seconds. After a final 10 minutes of 98° C., the reaction was held at 12° C. A QX200 droplet reader was used to stream and to detect the number of droplets with a positive reaction. The results were analyzed using QuantaSoft software, and the methylation signals of cfDNA are presented as copies/mL.

TABLE 1
Primers and probes for droplet digital
methylation-specific PCR

| | | | SEQ | |
|---|---|---|---|---|
| | Primer | | ID | Product |
| Gene | Type | Sequence | NO | Size |
| HOXA9 | F¹ | 5′-GGGTTTTAY⁵GAGG | 1 | 110 |
| | | YGTTAAATA-3′ | | |
| | R² | 5′-AAAACCCTAAACCA | 2 | |
| | | ACCTCC-3′ | | |
| | FAM³ | 5′-ACAAACGACGACGC | 3 | |
| | | TAACCGAACAC-3′ | | |
| SCT | F | 5′-TGTTGGGGTATTAY | 4 | 125 |
| | | GTTAGGA-3′ | | |
| | R | 5′-AACRTTCACCAACR⁶ | 5 | |
| | | AACTCAAC-3′ | | |
| | HEX⁴ | 5′-AAAAAACGCGCGACT | 6 | |
| | | CCAACGACTACTACA-3′ | | |
| KCNS2 | F | 5′-YGGTAAGTTTTAYGT | 7 | 107 |
| | | TATGG-3′ | | |
| | R | 5′-AACTATAACTACAAC | 8 | |
| | | AAAAATCAATAAAAA-3′ | | |
| | FAM | 5′-TCGTTAATACCCCAA | 9 | |
| | | TACTCGATCTCCTAA-3′ | | |
| BARHL2 | F | 5′-GTATTAATTTTYGAA | 10 | 119 |
| | | TAGGGAGA-3′ | | |
| | R | 5′-TCCAATACCAATTCA | 11 | |
| | | AACAACC-3′ | | |
| | HEX | 5′-TAATAAATAAAAATT | 12 | |
| | | TCCGCCCGCTCGATA-3′ | | |

¹F: Forward
²R: Reverse
³FAM: Carboxyfluorescein (FAM)-labeled probe
⁴HEX: Hexachlorofluorescein (HEX)-labeled probe
⁵Y: representing either cytosine (C) or thymine (T)
⁶R: representing either adenine (A) or guanine (G)

### Statistical Analysis

Differences in the methylation signals of individual methylated probes between cancer and normal subjects were examined using the Mann-Whitney U test. Five-fold cross validation logistic regression models were constructed to distinguish between cancer patients (n=74) and normal subjects (n=63). The methylation levels of the four identified probes and potential confounding factors including age, gender, and smoking status were included in the model generation. Five logistic models were generated. A methylation risk score was calculated using the final model constructed by averaging the coefficient estimates of individual models. The differences in methylation scores between specific clinical characteristics, including tumor TNM stages (according to the American Joint Commission on Cancer 8th edition), EGFR mutation status and metastatic sites, were compared using the Mann-Whitney U test (between 2 groups) or Kruskal-Wallis test (among 3 or more groups). A receiver operating characteristic (ROC) analysis was performed to assess the accuracy of methylation risk scores as well as methylation levels of individual probes. The area under the receiver operating characteristic curve (AUROC) was calculated to obtain a threshold for the best sensitivity and specificity of each marker for lung cancer diagnosis. Patients were grouped based on the initial scores of 4 probes and/or the methylation scores to compare PFS using the log-rank test, and data were presented as Kaplan-Meier curves. Graphical illustrations were created using R or Prism 8 (GraphPad Software Inc.). Longitudinal follow-ups of individual patients were presented as a swimmer plot and a spaghetti plot using the R package ggplot2. Generalized estimating equations (GEE) were used to analyze repeated measurements of methylation scores of each patient. The number of follow-up visits, disease progression (PD), and their interaction term were included in the model to estimate the marginal mean differences between PD and non-PD patient groups.

### Example 1: Marker Discovery from Genome-wide Methylation Data of Primary Lung Cancer Tissues

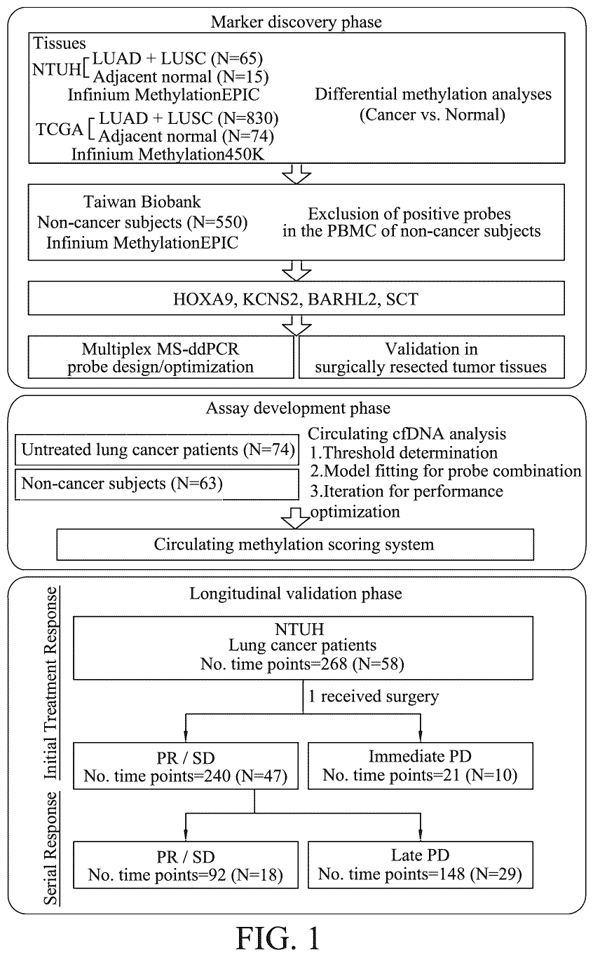

To identify cancer-specific methylated markers for blood-based assays, genome-wide methylation profiling was carried out on surgically resected lung cancer tissues from 65 lung cancer patients (30 adenocarcinomas and 35 squamous cell carcinomas) and 15 paired adjacent lung tissues from the National Taiwan University Hospital (NTUH) using Infinium MethylationEPIC BeadChips. Infinium HumanMethylation450 Methylation data of lung adenocarcinoma (N=460), squamous cell carcinoma (N=370), and adjacent lung tissues (N=74) were also obtained from The Cancer Genome Atlas (TCGA) for cross-ethnicity comparison. The criteria for the marker screening focused on candidate promoter probes with high methylation signals (0>0.5) in more than 50% of cancer tissues and low methylation signals (0>0.25) in more than 95% of the adjacent lung tissues. Since peripheral blood mononuclear cells (PBMCs) constitute a significant source of genomic DNA contamination in circulating cell-free DNA, the list of marker candidates is further refined by excluding probes with high methylation in the MethylationEPIC data of peripheral blood mononuclear cells (PBMCs) from 550 self-reported noncancer patients in the Taiwan Biobank. The different phases in discovering markers and developing and validating assays for characterizing disease state in a patient are depicted in FIG. 1.

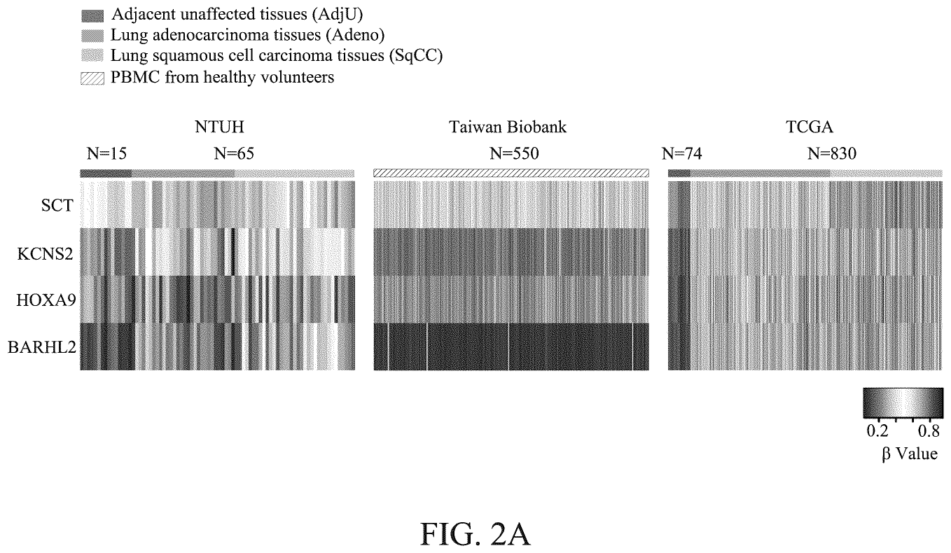

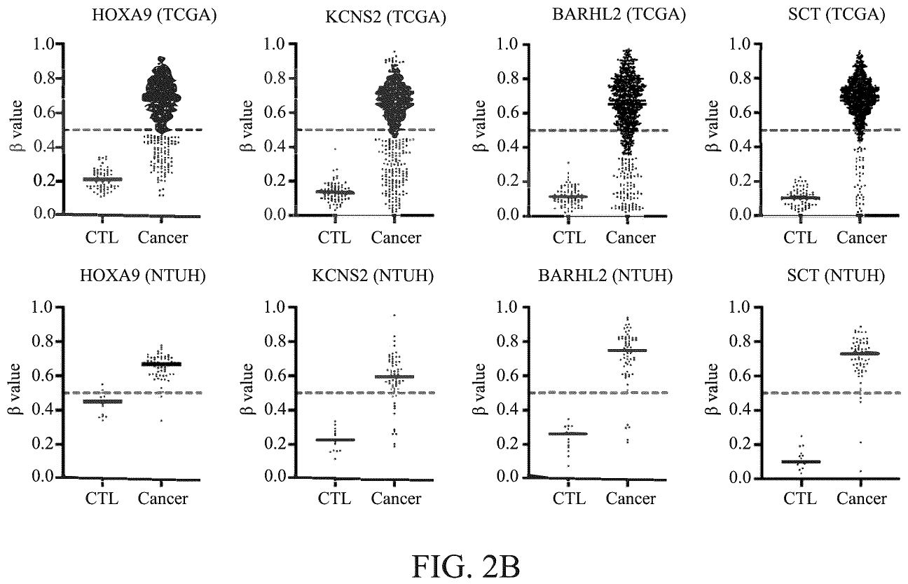

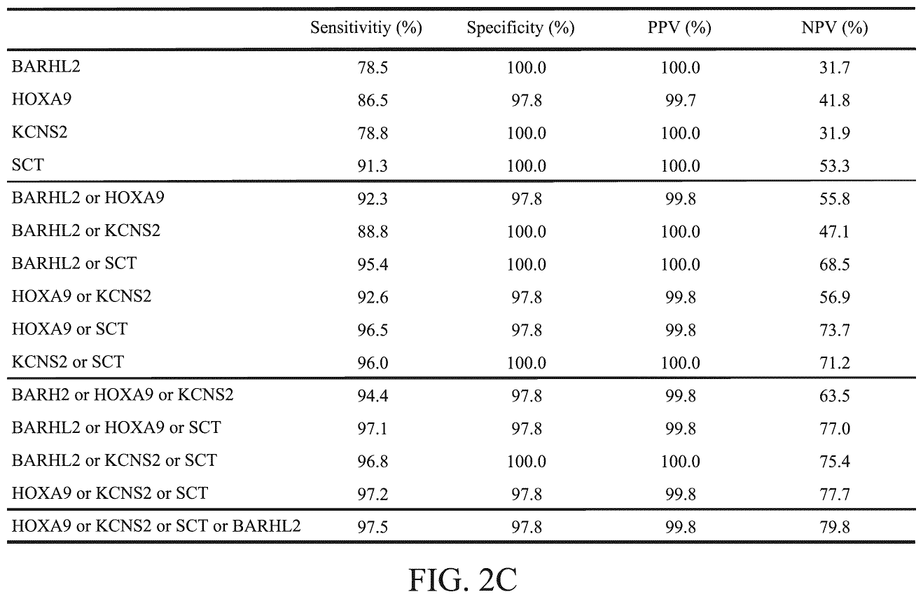

Iterations over all possible combinations were carried out with each combination comprising less than five probes. A combination of four promoter probes offered the optimal distinction between cancer and adjacent noncancer tissues in both NTUH and TCGA lung cancer cohorts. These probes were associated with homeobox A9 (HOXA9), potassium voltage-gated channel modifier subfamily S member 2 (KCNS2), BarH-like homeobox 2 (BARHL2) and secretin (SCT) (FIGS. 2A and 2B). High methylation (I>0.5) of any one of the four probes offers a positive predictive value (PPV) of 99.8% and a negative predictive value (NPV) of 79.8% in silico, as shown in FIG. 2C.

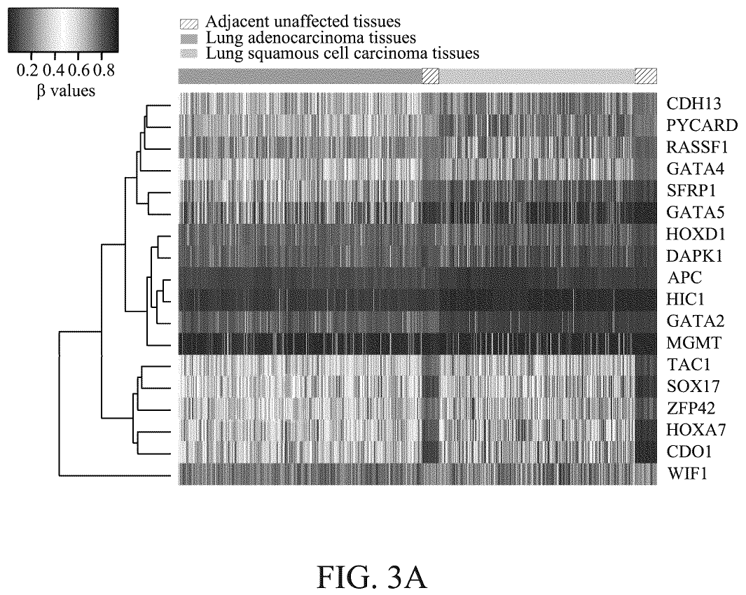

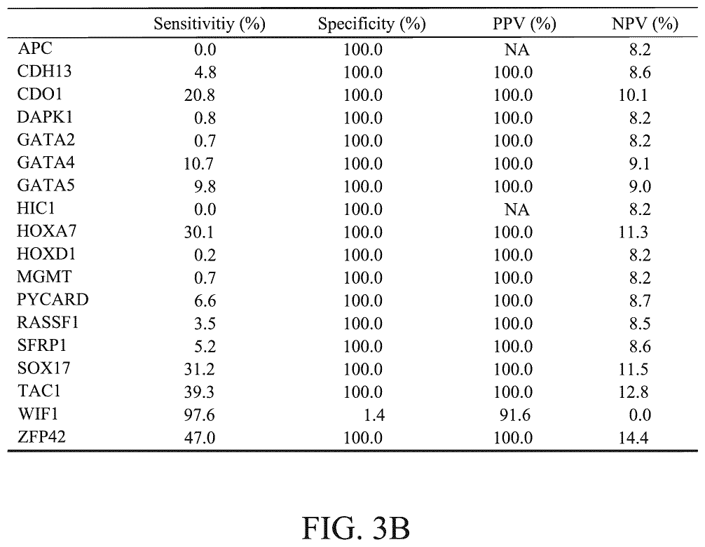

The sensitivities and specificities of previously reported cancer-specific marker genes in silico based on the TCGA cohort of lung cancer versus adjacent noncancer tissues were analyzed and compared, such as APC, CDH13, GATA4, GATAS, SOX17, and SFRP117-19. The promoter methylation status of most individual genes showed suboptimal sensitivities or negative predictive values in the TCGA cohort, as shown in FIG. 3.

### Example 2: Primers and Probes for Multiplex Methylation-specific Droplet Digital PCR (MS-ddPCR)

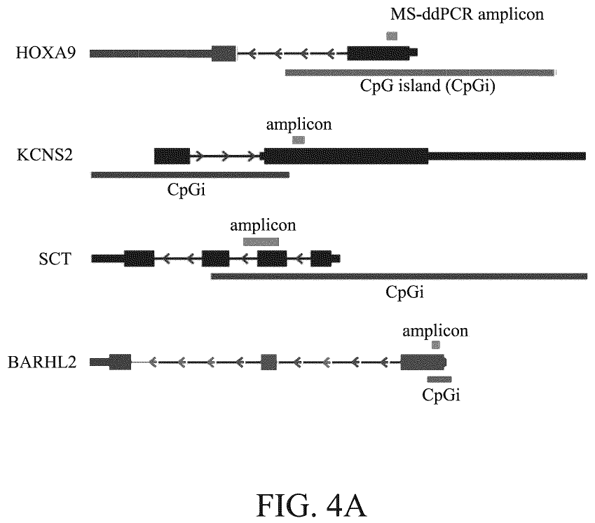

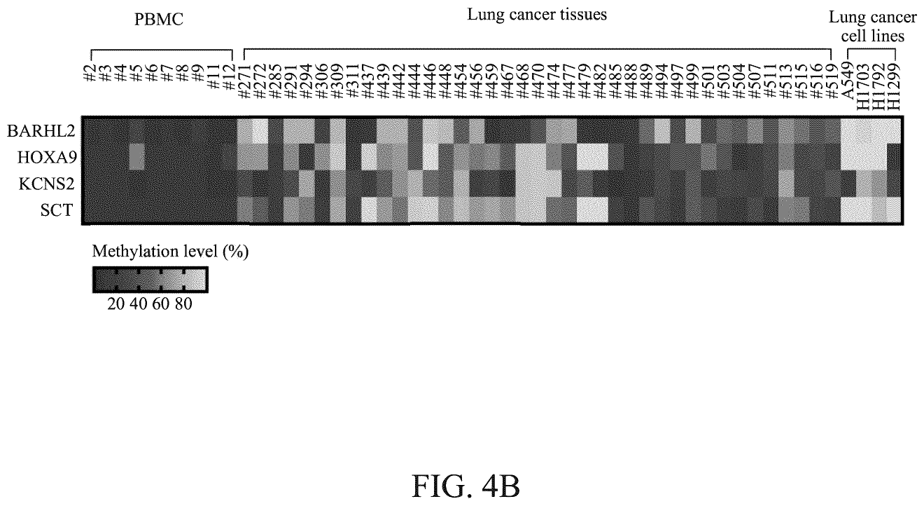

Specific primer and probe combinations based on the genomic locations of the identified MethylationEPIC probes for methylation-specific detection were designed and tested, as shown in FIGS. 4A and 4B. These customized primer and probe sets targeting the promoter regions of BARHL2, HOXA9, KCNS2, and SCT were tested in four lung cancer lines (A549, H1703, H1972, and H1299) and 39 surgically resected lung cancer tissues obtained at NTUH using quantitative methylation-specific PCR. The demographics of the archived surgically resected lung cancer tissues is shown in Table 2

TABLE 2
Demographics of the archived surgically resected lung cancer
tissues from the National Taiwan University Hospital (NTUH)

| | |
|---|---|
| | Lung Cancer tissues |
| Characteristics | (N = 39) |

Gender, n (%)

| | | |
|---|---|---|
| Male | 27 | (69.2%) |
| Female | 12 | (30.8%) |

Age, mean (range)
63.8 (31-46)
Smoking, n (%)

| | | |
|---|---|---|
| Never | 13 | (33.3%) |
| Active | 13 | (33.3%) |
| Former | 7 | (17.9%) |
| Not available | 6 | (15.4%) |

Tumor stage, n (%)

| | | |
|---|---|---|
| T1 | 7 | (17.9%) |
| T2 | 20 | (51.3%) |
| T3 | 5 | (12.8%) |
| T4 | 6 | (15.4%) |
| missing | 1 | (2.6%) |

Nodal stage, n (%)

| | | |
|---|---|---|
| N0 | 20 | (51.3%) |
| N1 | 5 | (12.8%) |
| N2 | 13 | (33.3%) |
| N3 | 1 | (2.6%) |

Stage, n (%)

| | | |
|---|---|---|
| Stage I | 17 | (43.6%) |
| Stage II | 3 | (7.7%) |
| Stage III | 18 | (46.2%) |
| Stage IV | 1 | (2.6%) |

All four lung cancer lines showed high methylation in at least two of the four probes. Moreover, consistent with the genome-wide methylation analysis for marker discovery, at least one probe of the four-probe panel showed high methylation in most primary lung cancer tissues as opposed to the PBMCs from the noncancer controls, as shown in FIG. 4B.

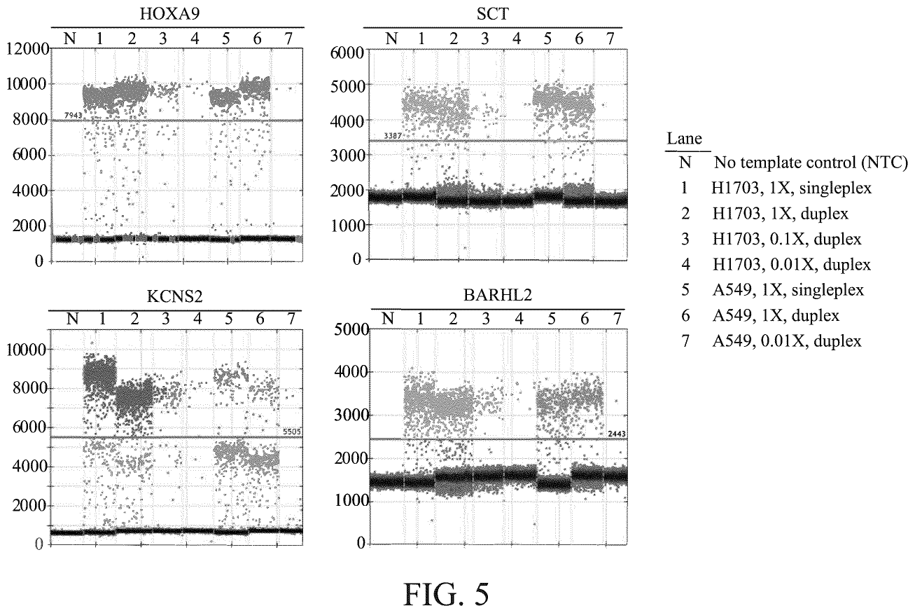

Next, owing to the low quantities of plasma cell-free DNA in general, the assay was further optimized for multiplex MS-ddPCR that showed comparable signals as the single-plex assays for individual markers in serial dilutions, as shown in FIG. 5.

### Example 3: Scoring System Based on Four Methylated Plasma cfDNA Markers

To evaluate the performance of the four-marker panel in the plasma of non-small cell lung cancer patients at presentation and follow-up visits for longitudinal monitoring of treatment responses, 74 patients with late-stage diseases and 63 self-reported healthy controls were prospectively enrolled at the National Taiwan University Hospital from Dec. 2015 to Nov. 2019. The demographics of the recruited individuals are shown in Table 3.

TABLE 3
Demographics of lung cancer patients
and noncancer control subjects

| | | |
|---|---|---|
| | Noncancer | |
| | Control | Lung Cancer |
| Characteristics | (N = 63) | (N = 74) |

Gender, n (%)

| | | | | |
|---|---|---|---|---|
| Male | 28 | (44.4%) | 34 | (45.9%) |
| Female | 35 | (55.6%) | 40 | (54.1%) |

Age, in years

| | | |
|---|---|---|
| Mean | 43.8 | 62.8 |
| Range | 22-75 | 42-81 |

Smoking, n (%)

| | | | | |
|---|---|---|---|---|
| Never | 52 | (82.5%) | 51 | (68.9%) |
| Active | 3 | (4.8%) | 16 | (21.6%) |
| Former | 6 | (9.5%) | 7 | (9.5%) |
| Not available | 2 | (3.2%) | 0 | (0%) |

Initial tumor diameter, in centimeters

| | |
|---|---|
| Median | 4.2 |
| Range | 1.5-11.3 |

Tumor stage, n (%)

| | | |
|---|---|---|
| T1 | 3 | (4.1%) |
| T2 | 19 | (25.7%) |
| T3 | 10 | (13.5%) |
| T4 | 42 | (56.8%) |

Nodal stage, n (%)

| | | |
|---|---|---|
| N0 | 14 | (18.9%) |
| N1 | 5 | (6.8%) |
| N2 | 19 | (25.7%) |
| N3 | 36 | (48.6%) |

Metastasis, n (%)

| | | |
|---|---|---|
| Lung to lung metastasis | 38 | (51.4%) |
| Brain metastasis | 29 | (39.2%) |
| Bone metastasis | 39 | (52.7%) |
| Pleural seeding/effusion | 28 | (37.8%) |
| Others | 17 | (23.0%) |

Stage, n (%)

| | | |
|---|---|---|
| Stage IIIB | 6 | (8.1%) |
| Stage IV | 68 | (91.9%) |

EGFR mutation, n (%)

| | | |
|---|---|---|
| Exon 19 deletion | 28 | (37.8%) |
| L858R | 27 | (36.5%) |
| Other mutations | 4 | (5.4%) |
| Wild type | 14 | (18.9%) |
| Not available | 1 | (1.4%) |

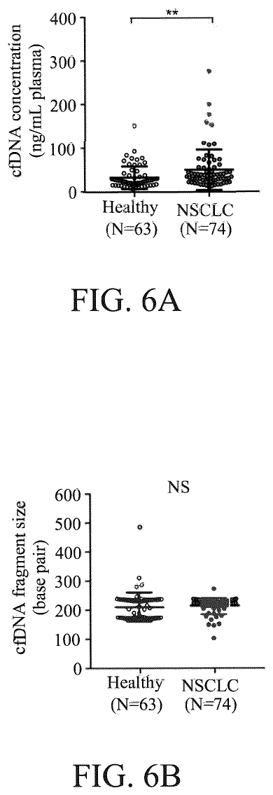

The peripheral blood samples were subjected to plasma cell-free DNA extraction and quantitative analysis of four methylated markers using multiplex MS-ddPCR. At the initial presentation before treatment, the cfDNA concentrations were significantly higher in NSCLC patients (median 35.1 ng/mL, range from 10.5 to 277.7 ng/mL) than in noncancer subjects (median 24.2 ng/mL, range from 5.4 to 152.1 ng/mL) (p=0.0004, Mann-Whitney test). In contrast, the fragment sizes of cfDNA did not significantly differ between the two groups, as shown in FIGS. 6A and 6B.

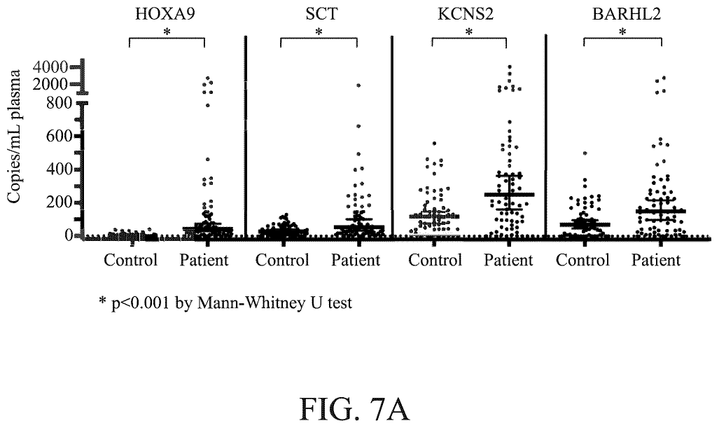

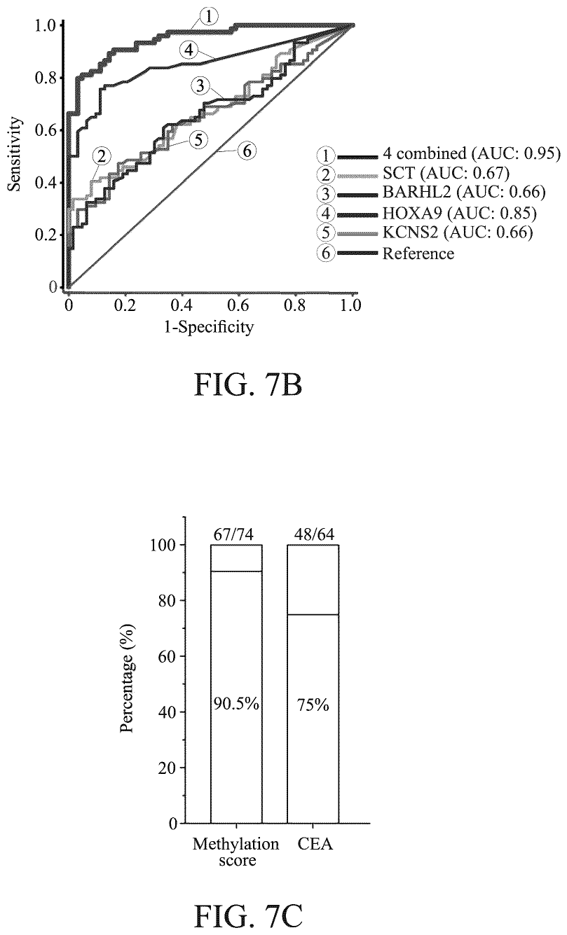

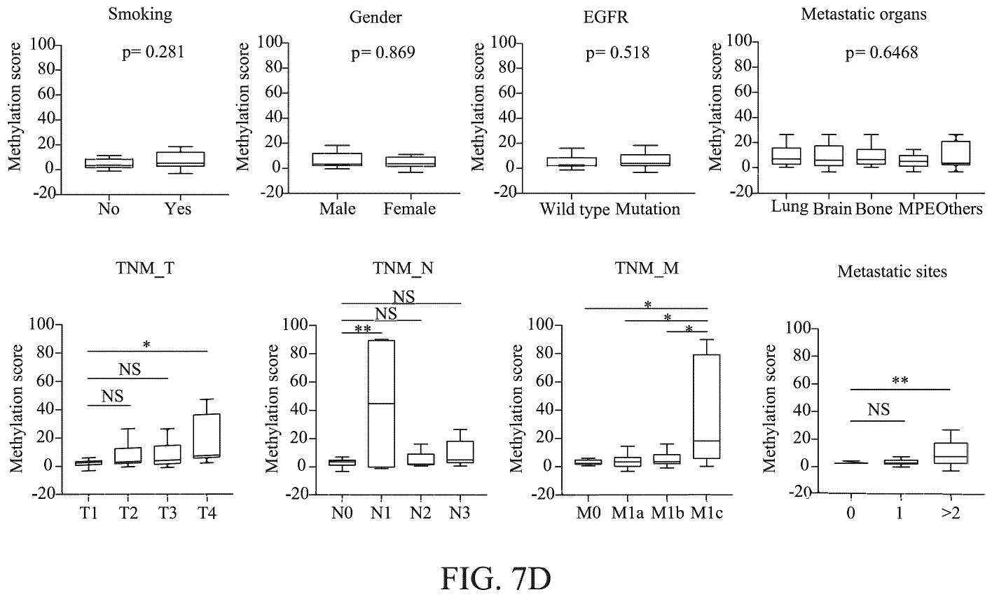

The median methylation signals of individual markers were significantly higher in lung cancer patients when compared to noncancer subjects, e.g., 16.7 copies/mL vs. 0.1 copies/mL in HOXA9 (p<0.05), 16.5 copies/mL vs. 0.1 copies/mL in SCT (p<0.05), 91.5 copies/mL vs. 0.1 copies/mL in KCNS2 (p<0.05), and 55.4 copies/mL vs. 0.1 copies/mL in BARHL2 (p <0.05), as shown in FIG. 7A.

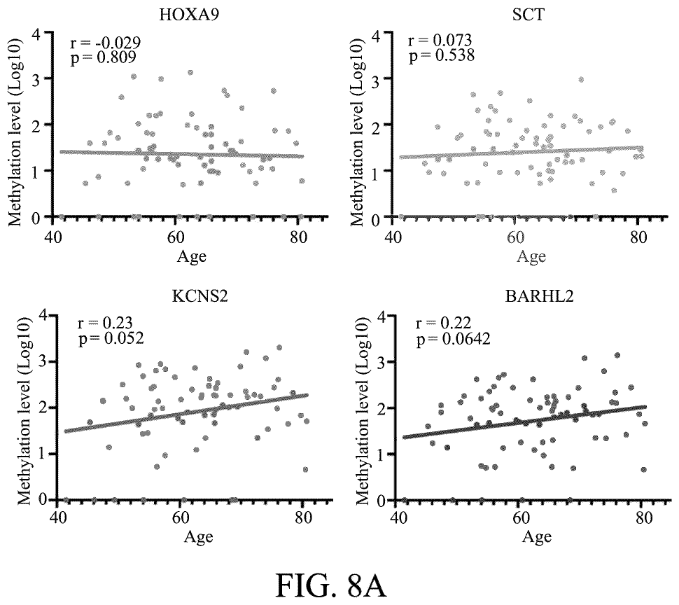

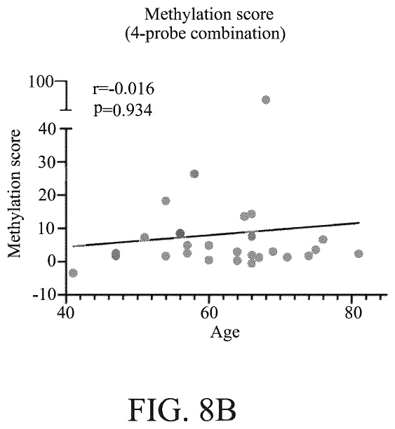

While age is a known factor that may affect DNA methylation, the methylation values of the identified markers did not appear to correlate with age, as shown in FIGS. 8A and 8B.

To determine the best combination of these markers for the distinction between cancer and noncancer at diagnosis, different logistic regression models were used to assign appropriate weights to individual markers and adjusted for potential confounders of DNA methylation, including age, gender, and smoking status. Samples taken at initial diagnosis before any treatment were used for model generation. Table 4 summarizes the results of different logistic regression models used, where Y is normal or cancer, X1, X2, X3, X4, X5, X6, X7, and X8 are age, gender, 2 dummy variables of smoking status of active smoker and former smoker, and methylation values of HOXA9, SCT, KCNS2, and BARHL2, respectively. Coefficient estimates of the variables and the corresponding statistics are also provided.

TABLE 4
Association model for lung cancer with
clinical DNA methylation variables

| | | | |
|---|---|---|---|
| Estimate | Std. Error | z value | p value |

Model 1: Logistic regression model
logit(P(Y = 1|X = x)) = −7.343 + 0.110X1 − 1.168X2 +
2.744X3 − 0.201X4 + 0.074X5 + 0.008X6 + 0.004X7 − 0.008X8

| | | | | |
|---|---|---|---|---|
| Intercept | −7.343 | 1.694 | −4.336 | <0.001 |
| X1 | 0.110 | 0.028 | 3.922 | <0.001 |
| X2 | −1.168 | 0.792 | −1.475 | 0.140 |
| X3 | 2.744 | 1.098 | 2.500 | 0.012 |
| X4 | −0.201 | 1.044 | −0.193 | 0.847 |
| X5 | 0.074 | 0.021 | 3.578 | <0.001 |
| X6 | 0.008 | 0.008 | 1.092 | 0.275 |
| X7 | 0.004 | 0.003 | 1.375 | 0.169 |
| X8 | −0.008 | 0.005 | −1.679 | 0.093 |

Model 2: Logistic regression model with 5-fold cross validation A
logit(P(Y = 1|X = x)) = −7.661 + 0.118X1 − 1.1420X2 +
2.427X3 − 0.096X4 + 0.083X5 + 0.009X6 + 0.003X7 − 0.009X8

| | | | | |
|---|---|---|---|---|
| Intercept | −7.661 | 2.001 | −3.829 | <0.001 |
| X1 | 0.118 | 0.034 | 3.500 | <0.001 |
| X2 | −1.420 | 0.939 | −1.512 | 0.130 |
| X3 | 2.427 | 1.158 | 2.097 | 0.036 |
| X4 | −0.096 | 1.154 | −0.083 | 0.934 |
| X5 | 0.083 | 0.024 | 3.467 | 0.001 |
| X6 | 0.009 | 0.007 | 1.210 | 0.226 |
| X7 | 0.003 | 0.003 | 1.227 | 0.220 |
| X8 | −0.009 | 0.005 | −1.824 | 0.068 |

Model 3: Logistic regression model with 5-fold cross validation B
logit(P(Y = 1|X = x)) = −7.445 + 0.106X1 − 0.468X2 +
3.310X3 − 0.336X4 + 0.062X5 + 0.002X6 + 0.003X7 − 0.003X8

| | | | | |
|---|---|---|---|---|
| Intercept | −7.445 | 1.904 | −3.910 | <0.001 |
| X1 | 0.106 | 0.030 | 3.502 | <0.001 |
| X2 | −0.468 | 0.843 | −0.555 | 0.579 |
| X3 | 3.310 | 1.370 | 2.415 | 0.016 |
| X4 | −0.336 | 1.062 | −0.317 | 0.752 |
| X5 | 0.062 | 0.021 | 2.923 | 0.003 |
| X6 | 0.002 | 0.009 | 0.173 | 0.862 |
| X7 | 0.003 | 0.003 | 0.953 | 0.341 |
| X8 | −0.003 | 0.006 | −0.474 | 0.636 |

Model 4: Logistic regression model with 5-fold cross validation C
logit(P(Y = 1|X = x)) = −6.878 + 0.107X1 − 1.463X2 +
2.205X3 − 0.264X4 + 0.067X5 + 0.010X6 + 0.004X7 − 0.008X8

| | | | | |
|---|---|---|---|---|
| Intercept | −6.878 | 1.762 | −3.903 | <0.001 |
| X1 | 0.107 | 0.030 | 3.562 | <0.001 |
| X2 | −1.463 | 0.840 | −1.742 | 0.082 |
| X3 | 2.205 | 1.155 | 1.909 | 0.056 |
| X4 | −0.264 | 1.032 | −0.256 | 0.798 |
| X5 | 0.067 | 0.021 | 3.204 | 0.001 |
| X6 | 0.010 | 0.008 | 1.244 | 0.214 |
| X7 | 0.004 | 0.003 | 1.362 | 0.173 |
| X8 | −0.008 | 0.005 | −1.537 | 0.124 |

Model 5: Logistic regression model with 5-fold cross validation D
logit(P(Y = 1|X = x)) = −7.036 + 0.108X1 − 1.189X2 +
2.527X3 − 0.690X4 + 0.072X5 + 0.005X6 + 0.005X7 − 0.010X8

| | | | | |
|---|---|---|---|---|
| Intercept | −7.036 | 1.900 | −3.703 | <0.001 |
| X1 | 0.108 | 0.032 | 3.318 | 0.001 |
| X2 | −1.189 | 0.889 | −1.337 | 0.181 |
| X3 | 2.527 | 1.262 | 2.002 | 0.045 |
| X4 | −0.690 | 1.193 | −0.578 | 0.563 |
| X5 | 0.072 | 0.023 | 3.153 | 0.002 |
| X6 | 0.005 | 0.011 | 0.509 | 0.611 |
| X7 | 0.005 | 0.003 | 1.445 | 0.148 |
| X8 | −0.010 | 0.007 | −1.481 | 0.139 |

Model 6: Logistic regression model with 5-fold cross validation E
logit(P(Y = 1|X = x)) = −9.156 + 0.131X1 − 1.581X2 +
4.261X3 − 1.115X4 + 0.109X5 + 0.012X6 + 0.004X7 − 0.009X8

| | | | | |
|---|---|---|---|---|
| Intercept | −9.156 | 2.405 | −3.807 | <0.001 |
| X1 | 0.131 | 0.038 | 3.422 | 0.001 |
| X2 | −1.581 | 1.135 | −1.393 | 0.164 |
| X3 | 4.261 | 1.588 | 2.683 | 0.007 |
| X4 | 1.115 | 1.710 | 0.652 | 0.514 |
| X5 | 0.109 | 0.037 | 2.981 | 0.003 |
| X6 | 0.012 | 0.012 | 1.014 | 0.310 |
| X7 | 0.004 | 0.003 | 1.094 | 0.274 |
| X8 | −0.009 | 0.007 | −1.370 | 0.171 |

Based on the above models, a methylation risk score was calculated as:

−7.635+0.114*age−1.224*gender+2.946*active smoking status−0.054*former smoking status+0.079*HOXA9+0.008*SCT+0.004*KCNS2−0.008*BARHL2.

With the methylation risk score calculated as above, the receiver operating characteristic (ROC) analysis estimated an AUC of 0.95 (95% confidence interval (CI), 0.92-0.98) with a sensitivity of 90.5% (95% CI, 81.7-95.3) and specificity 84.1% (95% CI, 73.2-91.1) at a cutoff score of 0.13 for the initial diagnosis of cancer. The AUC of individual markers are 0.67 for SCT, 0.66 for BARHL2, 0.85 for HOXA9 and 0.66 for KCNS2. The data are shown in FIG. 7B and in Table 5 below.

TABLE 5
Sensitivities and specificities of the four probes at
the cutoffs corresponding to the highest area under the
receiver operating characteristic (AUROC) curves for
the diagnosis of lung cancer versus non-cancer controls

| | Cutoff | Sensitivity | Specificity |
|---|---|---|---|
| HOXA9 | 6.3 | 77% | 87.3% |
| KCNS2 | 136 | 50% | 82.5% |
| BARHL2 | 70.6 | 54.1% | 74.6% |
| SCT | 23.9 | 51.4% | 68.3% |

Therefore, a methylation risk score greater than 0.13 may identify 90.5% of lung cancer patients (67/74). In contrast, carcinoembryonic antigen (CEA), a serum glycoprotein commonly used as a tumor marker in the clinic, was found to be elevated in only approximately 75% of lung cancer patients ( 48/64) in cohorts at the cutoff value of 5 ng/mL, as shown in FIG. 7C.

For the methylation risk score calculated above, it is found that methylation risk scores were independent of smoking status (p=0.281), gender (p=0.869), EGFR mutation status (p=0.517), and age (p=0.934) in the patients but highly correlated with tumor size and the number of metastatic sites, e.g., for tumor size more than 7 cm or with local invasion (T4) as well as for multiple metastatic sites (M1c) as shown in FIG. 7D. The results indicated that the methylation scores reflect tumor burden and disease severity.

### Example 4: Longitudinal Assessment of Methylation Risk Scores in the cfDNA at Serial Follow-ups

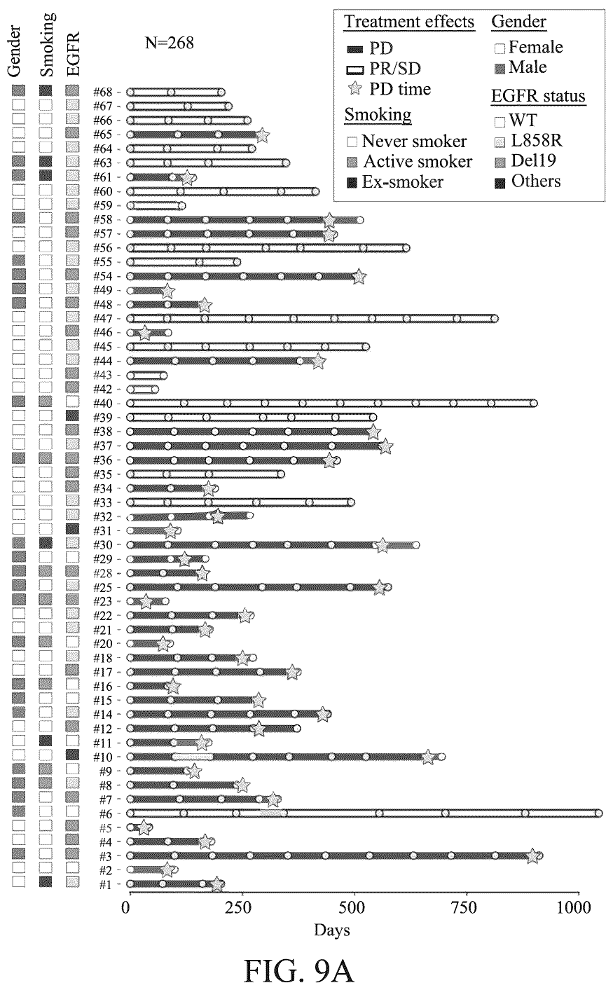

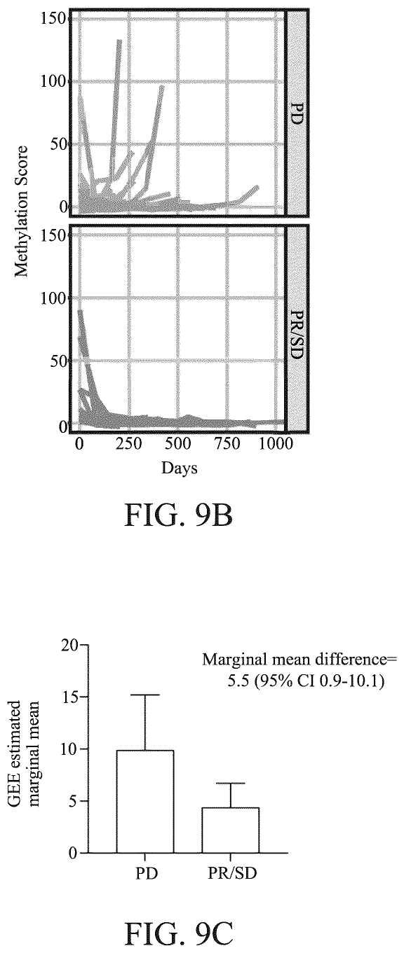

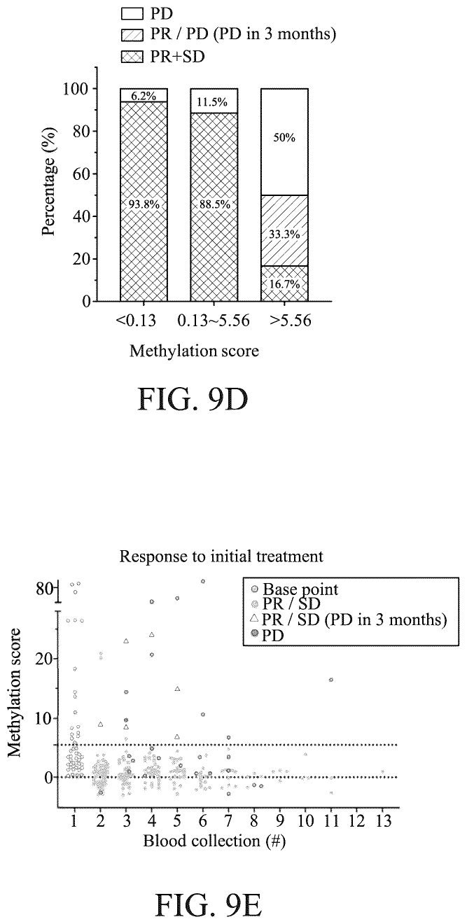

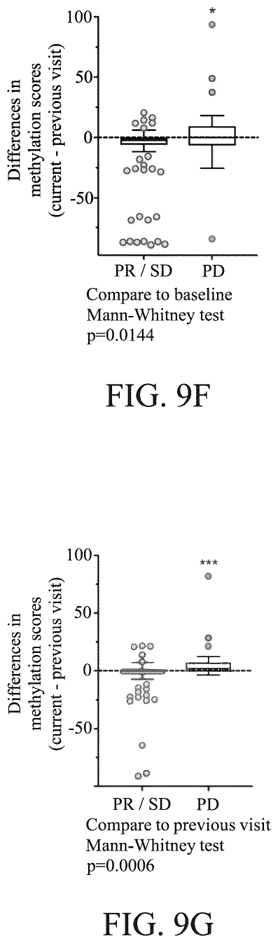

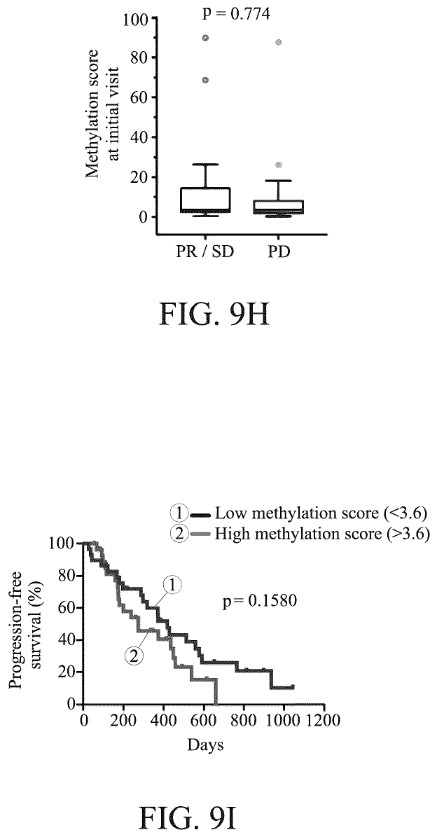

The clinical utility of methylation risk scores is assessed in the longitudinal follow-ups. cfDNA samples were obtained from non-small cell lung cancer patients every three months until disease progression (enlarged primary tumor or presence of new metastasis) according to the RECIST (Response Evaluation Criteria in Solid Tumors) criteria version 1.1. From January 2016 to November 2019, a total of 268 serial blood samples from 58 lung cancer patients were collected at the initial presentation and follow-up visits, the demographics and treatment effects are shown in FIG. 9A.

All patients received standard-of-care treatments, of which 47 received tyrosine kinase inhibitors; 6 received cisplatin and pemetrexed; 3 received carboplatin and pemetrexed; 1 received concurrent chemoradiotherapy (CCRT); and 1 received surgery. Based on the treatment responses, the patients were divided into three groups: immediate disease progression within the first three months (immediate PD, N=10), partial response or stable disease (PR/SD, N=18), and late disease progression after initial PR/SD (late PD, N=29). For clinical evaluation of the marker panel, patients whose initial methylation scores were above the cancer cutoff of 0.13 and who were followed up for at least six months were included in the analysis.

A significant reduction in methylation risk scores were observed following initial systemic treatments in the PR/SD group. On the other hand, the methylation scores were initially decreased but elevated when patients experienced disease progression (late PD), as shown in FIG. 9B. The generalized estimating equations (GEE) approach was applied to adjust for repeated measurements from each visit. As shown in FIG. 9C, a trend of a higher estimated marginal mean was found in the PD group (9.9 vs. 4.4, marginal mean difference 5.5, 95% CI 0.9-10.1), and the GEE model showed a difference (exp of 0) of 1.4 (95% CI 1.1-1.8) in the elevated methylation score in the PD group when considering the effect of repeated visits (interaction term of PD and visits), as shown in Table 6 below.

TABLE 6
Generalized estimated equations (GEE) model for longitudinal assessment
of methylation risk scores at the initial presentation and follow-up
visits in lung cancer patients with progressive disease (PD) vs.
those with partial response or stable disease (PR/SD)

| | | | | | 95% Wald |
|---|---|---|---|---|---|
| | | | | | CI for |
| Parameter | β | SE | p | Exp(B) | Exp(B) |
| (Intercept) | 2.548 | 0.4882 | <0.001 | 12.8 | 4.9-33.3 |
| PD | −0.222 | 0.5516 | 0.687 | 0.8 | 0.3-2.4 |
| Without PD | reference | — | — | 1.0 | — |
| Visit | −0.328 | 0.099 | 0.001 | 0.7 | 0.6-0.9 |
| PD* Visit | 0.319 | 0.1247 | 0.011 | 1.4 | 1.1-1.8 |
| Without PD* Visit | reference | — | — | 1.0 | — |

PD* Visit: The interaction between disease progression and each physician office visit

Moreover, to find a clinically practical threshold for daily practice, a percentile-based approach was undertaken to rank the methylation scores and to evaluate their correlation with disease status at each individual visit. In addition to the abovementioned diagnostic cutoff of 0.13 (FIG. 7B), it was determined that 5.56 was the value that hints at the potential for disease progression. It was found that 93.8% of patients with methylation scores below 0.13 and 88.5% of patients with methylation scores between 0.13 and 5.56 had achieved PR/SD at the time of blood draw, as shown in FIG. 9D. Nearly 83.3% of patients with methylation scores greater than 5.56 demonstrated progressive disease either at the time of the visit or in three months.

In other words, one-third of patients with high methylation scores showed molecular progression in the circulation at least three months earlier than imaging/clinical progression, as shown in FIG. 9E.

Likewise, the differences between methylation scores at individual follow-up visits and the respective baselines at the time of diagnosis were consistent with the clinical responses (PR/SD vs. PD, p=0.0144), as shown in FIG. 9F. Furthermore, the visit-by-visit methylation scores also tracked the dynamics of disease status, as the differences in methylation scores at two consecutive visits showed trends consistent with PR/SR or PD responses at each visit (p =0.0006), as shown in FIG. 9G.

On the other hand, the methylation scores at initial visits did not significantly differ between the PR/SD and PD groups (p=0.774, Mann-Whitney test), as shown in FIG. 9H. Although patients with higher methylation scores at baseline had a relatively shorter median progression-free survival (PFS) (273 days vs. 419 days), the trend did not reach statistical significance (p=0.16, by log-rank test), as shown in FIG. 9I.

These results suggested a promising role of methylation risk scores in serial disease monitoring instead of one-time prognostic stratification.

### Example 5: Application of Methylation Risk Scores in Disease Monitoring of EGFR Wild-type and Mutated Lung Cancers

The clinical utility of methylation risk scores was assessed for disease monitoring in lung cancer patients with or without specific driver mutations and receiving different therapies. The dynamic changes of methylation risk scores along the treatment course were examined in individual patients.

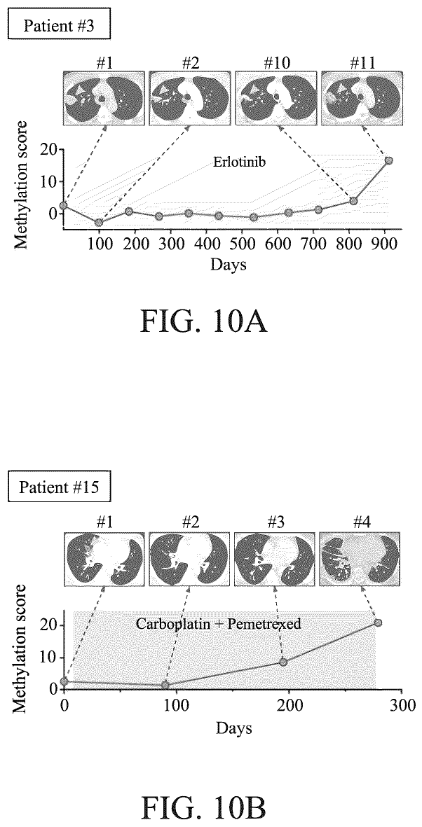

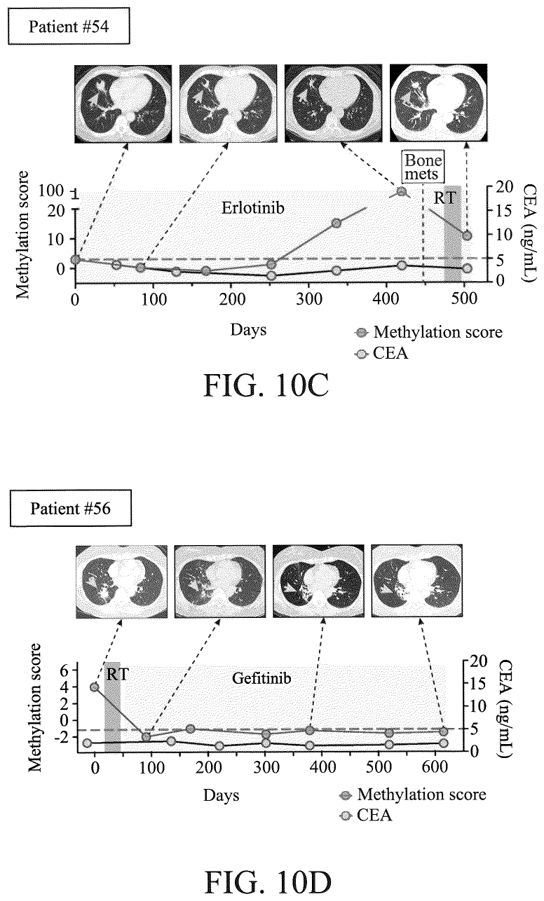

Patient #3 was a 46-year-old patient diagnosed with stage 4 lung adenocarcinoma carrying EGFR exon 19 deletions. He received erlotinib and soon achieved a partial response. His disease was under control for approximately 800 days, with the methylation risk scores remaining low at follow-up visits every three months. At the 10th blood sampling, a trend of methylation score going up for two consecutive visits was noticed before clinical evidence of progression. Three months later, computed tomography finally showed enlargement of the primary tumor in the right upper lobe accompanied by a further increase in the methylation score of 16.5, as shown in FIG. 10A.

Patient #15 was a 56-year-old patient presenting with stage 4 lung adenocarcinoma negative for major driver mutations. He achieved a partial response under carboplatin and pemetrexed treatment accompanied by decreases in circulating methylation scores. Nevertheless, despite the continued radiographic response, an elevation of methylation scores to 8.51 at 195 days was noticed. Clinical progression was eventually evident at 279 days, approximately three months later, as shown in FIG. 10B.

Compared with commonly used serum markers such as carcinoembryonic antigen (CEA), methylation scores appeared to be more sensitive in detecting disease progression.

For patient #54, a 69-year-old patient with stage 4 lung adenocarcinoma receiving erlotinib treatment, the methylation scores decreased initially but increased again at approximately one year despite the shrinkage of the primary lung tumor on chest tomography. The methylation scores continued to rise until new bone metastases (bone mets) at the lumbar spine and pelvis were noted six months later, indicating that the elevation of methylation scores preceded evident radiographic and clinical progression.

The CEA levels remained low throughout the entire course. It was noted that the methylation score declined rapidly after local palliative radiotherapy (RT), shown in FIG. 10C.

On the other hand, a 48-year-old patient with stage IIIb lung adenocarcinoma received concurrent chemoradiotherapy (CCRT) and gefitinib as shown in FIG. 10D. The patient's methylation scores decreased significantly following CCRT and remained low for at least 600 days with no signs of molecular progression.

These examples demonstrated the clinical utility of the circulating 4-gene methylation scores in disease monitoring for different molecular subtypes of non-small cell lung cancer. For example, clinical applications of this assay are feasible in patients who received either chemotherapy or targeted therapy.

### Example 6: Genome-wide DNA Methylation Measurement of Surgically Resected Lung Cancer Tissues in Cohort 2

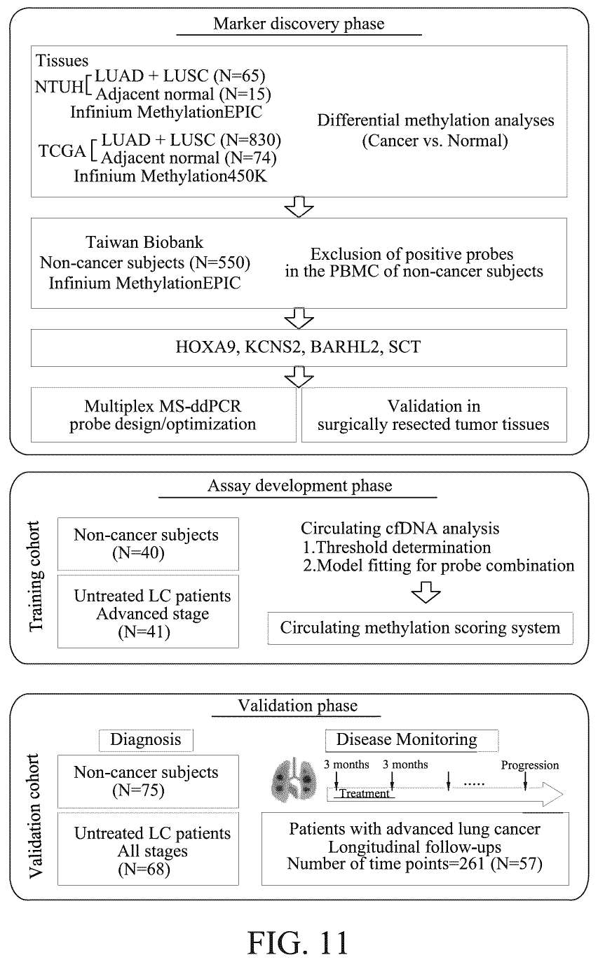

A similar flow as shown in Example 1 was used, but with a training cohort and a validation cohort, as shown in FIG. 11. In cohort 2, sixty-five surgically resected lung tumor tissues (adenocarcinoma, N=30; squamous cell carcinoma, N=35) and/or the adjacent unaffected tissues (N=15) from patients with non-small cell lung cancer were collected at National Taiwan University Hospital (NTUH cohort) from Sep 1994 to Oct 2011. Informed consent was obtained prior to study enrollment for each patient. DNA extraction of primary tissues was performed using the traditional phenol chloroform method. The extracted DNA was subjected to bisulfite treatment using the EZ DNA Methylation kit (Zymo Research, Irvine, CA) followed by genome-wide methylation measurement using the Infinium MethylationEPIC Arrays (Illumina, San Diego, CA). Raw intensity data were obtained as IDAT files and processed using the R package minfiv1.30.016. The methylation data were uploaded to the Gene Expression Omnibus (GEO, RRID:SCR 005012) with the accession number GSE159350. The part of study was approved by the Institutional Review Board (IRB) of NTUH (201701010RINB and 201605088RIND).

### Enrollment of Lung Cancer Patients for Plasma Cell-free DNA Analysis

Patients over 20 years old who were newly diagnosed with non-small cell lung cancer at National Taiwan University Hospital (NTUH) from Dec 2015 to Dec 2020 were prospectively enrolled. Pregnant or lactating women were excluded. Non-cancer subjects over 20 years old were enrolled at the Health Management Center of NTUH and Far Eastern Memorial Hospital (FEMH). Informed consent from each subject was obtained prior to the study enrollment. For each participant in the training or the validation cohort, 10 mL of peripheral blood was collected before treatment. Demographic data and clinical information, including age, gender, smoking status, clinical stages, histological diagnosis, metastatic sites, mutation status, and treatment regimens, were recorded. For longitudinal follow-up of each patient, blood was collected before treatment and every three months until disease progression or death. Medical records and images of each patient were independently reviewed by two chest physicians to determine the status of disease progression (PD), partial response (PR), or stable disease (SD) at each follow-up visit during the treatment course until disease progression, change of treatment regimen or drop out of the study. The part of study was approved by the Institutional Review Board of NTUH (IRB#201510099RINC) and FEMH (IRB#FEMH-IRB-107174-F).

### Statistical Analysis

Clinical characteristics between cancer and non-cancer cases in the training and the validation cohorts were compared using the Mann-Whitney U test, t-test, or chi-squared test as suitable. A logistic regression model based on the methylation levels of the four probes was used to calculate methylation risk scores. The differences in methylation scores between clinical characteristics were compared using the Mann-Whitney U test (between 2 groups) or Kruskal-Wallis test (between 3 or more groups). A multivariable logistic regression model was used to assess the independence of methylation score in predicting cancer after adjusting for age, gender, and parameters with p<0.1 in the univariable analysis. Receiver operating characteristic (ROC) analyses were used to assess the performance of methylation risk scores and methylation levels of individual probes. Longitudinal follow-ups of individual patients were presented as a swimmer plot and a spaghetti plot using the R package ggplot2 (RRID:SCR 014601). Clinical characteristics between patients with partial response/stable diseases and those with progressive diseases were compared using the Mann-Whitney U test or chi-squared test. Generalized estimating equations (GEE) were used to analyze repeated measurements of methylation scores of each patient.

Marker Discovery from Genome-wide Methylation Data of Primary Lung Cancer Tissues

To identify cancer-specific methylated markers for blood-based assays, genome-wide methylation profiling was performed on surgically resected lung cancer tissues from 65 lung cancer patients (30 adenocarcinomas and 35 squamous cell carcinomas) and 15 paired adjacent lung tissues from National Taiwan University Hospital (NTUH) using Infinium MethylationEPIC BeadChips. Infinium HumanMethylation450 Methylation data of lung adenocarcinoma (N=460), squamous cell carcinoma (N=370), and adjacent lung tissues (N=74) was also obtained from The Cancer Genome Atlas (TCGA) for cross-ethnicity comparison. Candidate promoter probes with high methylation signals (0>0.5) in more than 50% of cancer tissues and low methylation signals (0<0.25) in more than 95% of the adjacent lung tissues were focused. Since peripheral blood mononuclear cells (PBMCs) constitute a significant source of genomic DNA contamination in circulating cell-free DNA, the list by excluding probes with high methylation levels in the PBMCs from 550 self-reported non-cancer patients in Taiwan Biobank (www.twbiobank.org.tw) was further refined.

To simplify assay complexity for routine clinical uses, iterations over all possible combinations composed of less than five probes were performed, and a combination of four promoter probes that offered the optimal distinction between cancer and adjacent non-cancer tissues in both NTUH and TCGA lung cancer cohorts was identified. These probes were associated with homeobox A9 (HOXA9), potassium voltage-gated channel modifier subfamily S member 2 (KCNS2), BarH-like homeobox 2 (BARHL2), and secretin (SCT). High methylation (0>0.5) of any one of the four probes offers a positive predictive value (PPV) of 99.8% and a negative predictive value (NPV) of 79.8% in silico.

Develop a Scoring System Based on Four Methylated Plasma cfDNA Markers

Subsequently, specific primer/probe combinations for multiplex methylation specific droplet digital PCR (MS-ddPCR) were designed. These customized primer/probe sets shown in FIG. 4A targeting the promoter regions of BARHL2, HOXA9, KCNS2, and SCT in 39 surgically resected lung adenocarcinoma tissues obtained at NTUH and in four lung cancer lines (A549, H1703, H1972, and H1299) were tested. Consistent with the genome-wide methylation analysis, at least one of the four probes showed high methylation in most primary lung adenocarcinoma tissues.

Next, non-small cell lung cancer patients were enrolled at National Taiwan University Hospital to evaluate the performance of the four-marker panel in the plasma. In the training cohort, forty-one lung adenocarcinoma patients with advanced diseases and 40 self-reported healthy controls were enrolled. Table 7 shows the demographics of non-small cell lung cancer patients and non-cancer controls in the training and validation cohorts.

TABLE 7
Demographics of non-small cell lung cancer patients and non-cancer controls in the training and validation cohorts

| | Training cohort | | | Validation cohort | | |
|---|---|---|---|---|---|---|
| | Non-cancer | Advanced | | Non-cancer | All stage | |
| | case | lung cancer | | case | lung cancer | |
| Characteristics | (N = 40) | (N = 41) | p value | (N = 75) | (N = 68) | p value |
| Gender, N (%) | | | | | | |
| Male | 20 (50.0%) | 20 (48.8%) | 0.913 | 35 (46.7%) | 36 (52.9%) | 0.963 |
| Female | 20 (50.0%) | 21 (51.2%) | | 40 (53.3%) | 32 (47.1%) | |
| Age, in years | | | | | | |
| Mean | 50.8 | 61.4 | <0.0001 | 47.5 | 61.4 | <0.0001 |
| Range | 24~70 | 42~81 | | 22~78 | 33~89 | |
| Smoking, N (%) | | | | | | |
| Never | 34 (85.0%) | 26 (63.4%) | 0.04 | 60 (80%) | 54 (79.4%) | 0.833 |
| Active | 3 (7.5%) | 12 (29.3%) | | 7 (9.3%) | 9 (13.2%) | |
| Former | 3 (7.5%) | 3 (7.3%) | | 8 (10.7%) | 5 (7.4%) | |
| cfDNA conc., ng/mL | | | | | | |
| Median | 21.6 | 28.5 | 0.028 | 22.3 | 28.2 | 0.027 |
| Range | 5.4~93.6 | 11.1~201.6 | | 8.2~152.1 | 7.4~279.0 | |
| cfDNA fragment size, base pairs | | | | | | |
| Median | 175 | 216 | 0.056 | 175 | 179 | 0.269 |
| Range | 168~485 | 105238 | | 161~312 | 163~275 | |
| Stage, N (%) | | | | | | |
| In situ | | 0 | | | 3 (4.4%) | |
| I | | 0 | | | 29 (42.6%) | |
| II | | 0 | | | 1 (1.5%) | |
| III | | 1(2.4%) | | | 7 (10.3%) | |
| IV | | 40 (100%) | | | 28 (41.2%) | |
| EGFR status, N (%) | | | | | | |
| Exon 19 Del | | 16 (39.0%) | | | 12(17.6%) | |
| L858R | | 12 (29.3%) | | | 15 (22.1%) | |
| Others | | 1 (2.4%) | | | 3 (4.4%) | |
| Wild type | | 9 (29,3%) | | | 5 (7.4%) | |
| Not Available | | 0 (0%) | | | 33(48.5%) | |

Before any treatment, the cfDNA concentrations were significantly higher in lung cancer patients (median 28.5 ng/ml, 11.1 to 201.6 ng/mL) than in noncancer subjects (median 21.6 ng/mL, range, 5.4 to 93.6 ng/mL) (p=0.028, Mann-Whitney test). There were no statistically significant differences in gender and cfDNA fragment sizes between the two groups. A methylation risk scoring system was developed by constructing a logistic regression model to assign appropriate weights to individual markers. The methylation risk score was calculated as −1.547+0.024*SCT+0.084*HOXA9-0.039*BARHL2+0.031*KCNS2.

### Methylation Risk Scores Identify Lung Cancer Patients at All Stages

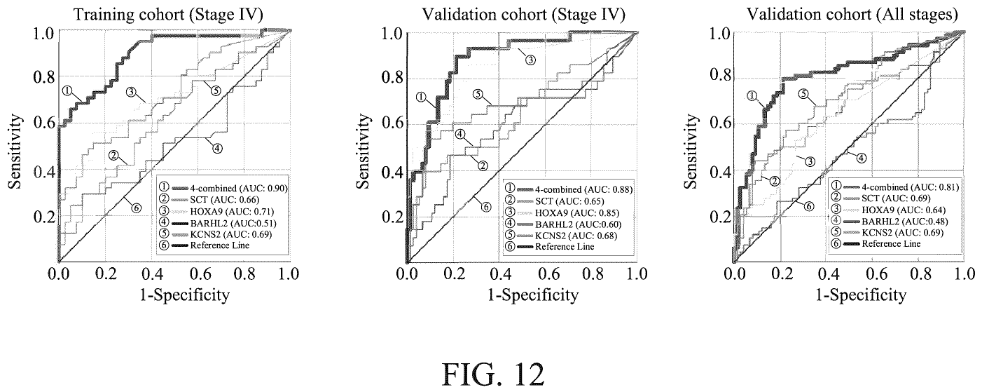

In the training cohort, the receiver operating characteristic (ROC) analysis of methylation risk scores estimated an area under the curve (AUC) of 0.90 (95% C.I.=0.83-0.97) was superior to those of individual markers (SCT: 0.68, BARHL2: 0.54, HOXA9: 0.80, and KCNS2: 0.73) as shown in FIG. 12. With a cutoff value of −0.764, the methylation score achieved a sensitivity of 90.2% and a specificity of 70.0% for lung cancer diagnosis with a positive predictive value of 75.5% and a negative predictive value of 87.5%. The numbers outperformed the similar measures calculated using other cutoff values shown in Table 8.

TABLE 8
Comparison of sensitivity, specificity, positive predictive value
(PPV) and negative predictive value (NPV) of methylation scores
at various cut-off values for the diagnosis of lung cancer

| | Cut-off | | | | |
|---|---|---|---|---|---|
| | values | Sensitivity | Specificity | PPV | NPV |
| Training cohort | 0.764 | 90.2% | 70.0% | 75.5% | 87.5% |
| | −0.488 | 85.4% | 75.0% | 77.8% | 83.3% |
| | 0.268 | 73.2% | 82.5% | 81.1% | 75.0% |
| Validation cohort | −0.764 | 88.2% | 38.7% | 56.6% | 78.4% |
| Total cohort | | | | | |
| Early stages (I and II) | −0.764 | 81.8% | 49.6% | 31.8% | 90.5% |
| Late stages (III and IV) | −0.764 | 92.1% | 49.6% | 54.7% | 90.5% |
| All stages | −0.764 | 89.0% | 49.6% | 62.6% | 82.6% |

Given the methylation scoring system was derived from only patients with advanced diseases, this system was applied in an independent validation cohort of 68 lung cancer patients at all stages versus 75 non-cancer controls for potentially broader applicability, especially for early-stage lung cancer since few tests were available for patients in this category, as shown in Table 7.

The AUC of methylation scores was calculated as 0.88 (95% C.I.=0.85-0.95) for patients with advanced diseases in the validation cohort. As shown in FIG. 12, the overall AUC still achieved 0.81 (95% C.I.=0.73-0.88) for patients at all stages. The multivariable analysis revealed that methylation score greater than −0.764 was an independent predictor for lung cancer diagnosis in either training (odds ratio, 80.5, p=0.001) or validation cohorts (odds ratio, 3.15, p =0.019) after adjusting for age, gender, smoking status, cfDNA fragment size and concentration, as shown in Table 9.

TABLE 9
Multivariable logistic regression analysis for
the diagnosis of non-small cell lung cancer

| Variables | OR | p value | 95% C.I. |
|---|---|---|---|
| Training cohort | | | |
| Methylation score > −0.764 | 80.50 | 0.001 | 6.725-963.575 |
| Age over 65 | 2.58 | 0.268 | 0.482-13.833 |
| Gender (male) | 0.45 | 0.305 | 0.101-2.052 |
| Active smoker vs. non-smoker | 58.49 | 0.007 | 3.069-1114.759 |
| Ex-smoker vs. non-smoker | 1.25 | 0.845 | 0.129-12.192 |
| cfDNA size | 1.01 | 0.114 | 0.997-1.026 |
| cfDNA concentration | 1.02 | 0.260 | 0.985-1.057 |
| Validation cohort | | | |
| Methylation score > −0.764 | 3.15 | 0.019 | 1.207-8.242 |
| Age over 65 | 3.30 | 0.012 | 1.302-8.385 |
| Gender (male) | 0.87 | 0.744 | 0.376-2.012 |
| Active smoker vs. non-smoker | 1.62 | 0.430 | 0.488-5.377 |
| Ex-smoker vs. non-smoker | 0.66 | 0.553 | 0.167-2.603 |
| cfDNA size | 1.01 | 0.233 | 0.995-1.02 |
| cfDNA concentration | 1.01 | 0.283 | 0.993-1.024 |

OR: Odds ratio;
C.I.: Confidence interval

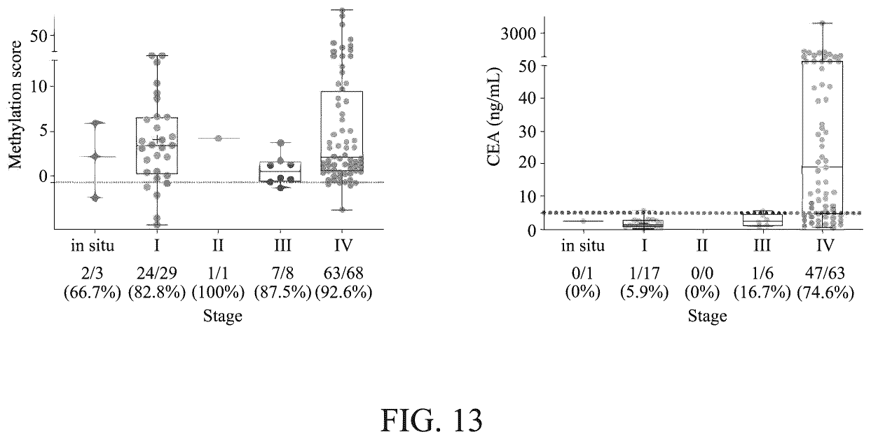

Overall, in the combined training/validation cohorts, the assay achieved a sensitivity of 82.8% and 92.6% for patients with stage I and IV diseases, respectively, as shown in FIG. 13. By contrast, carcinoembryonic antigen (CEA), a serum glycoprotein commonly used as a tumor marker in the clinic, was hardly elevated (cutoff value, 5 ng/mL) in early-stage diseases, also shown in FIG. 13. In addition, higher methylation scores were observed in late-stage diseases (mean=8.43, S.D.=18.14) as compared with early-stage diseases (mean=3.89, S.D.=5.38)(Mann-Whitney test, p=0.049). The data indicate that methylation scores correlate with disease burden.

Longitudinal Assessment of Methylation Risk Scores in cfDNA at Serial Follow-ups

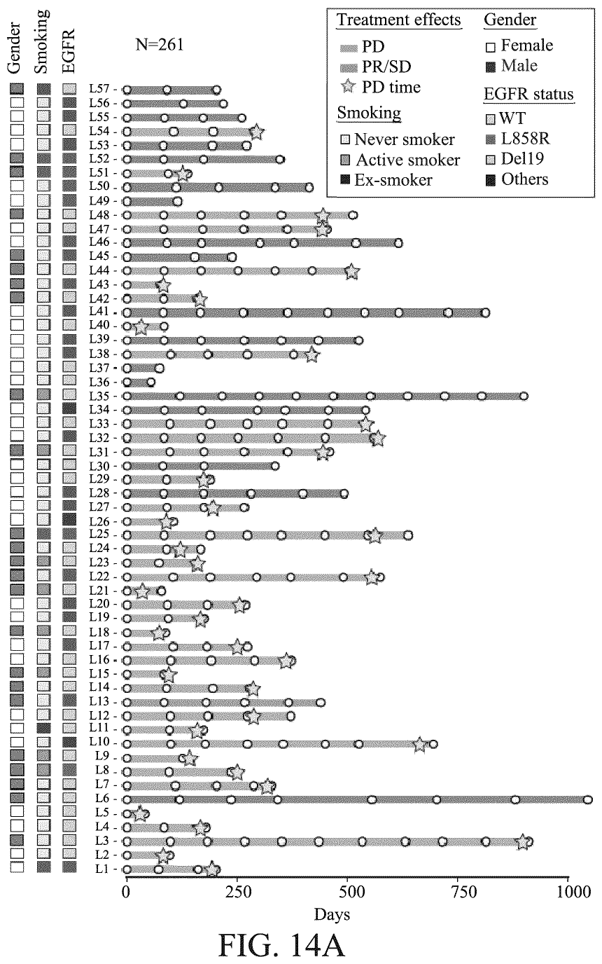

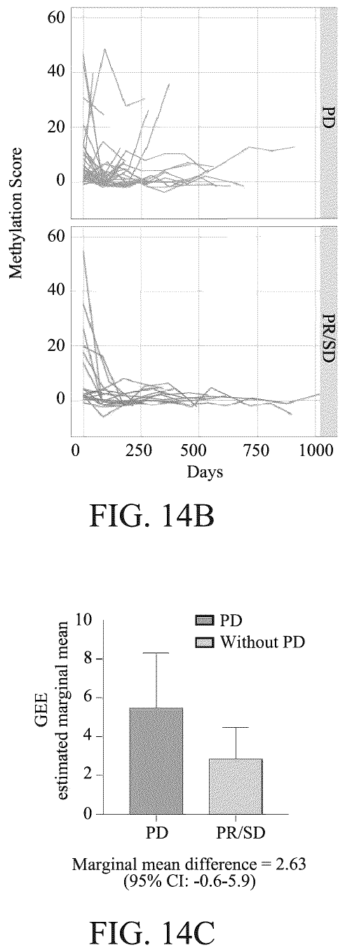

Subsequently, the dynamic changes of methylation risk scores was evaluated in a longitudinal patient cohort for disease status monitoring. cfDNA samples were obtained from advanced lung adenocarcinoma patients every three months until disease progression (e.g., enlarged primary tumor or presence of new metastasis) according to the RECIST (Response Evaluation Criteria in Solid Tumors) criteria version 1.1. From January 2016 to December 2020, FIG. 14A shows a total of 261 serial blood samples from 57 lung cancer patients were collected at initial presentations and follow-up visits. All patients received standard-of-care treatments (47 received tyrosine kinase inhibitors; 6 received cisplatin and pemetrexed; 3 received carboplatin and pemetrexed; 1 received concurrent chemoradiotherapy). Based on the treatment responses, the patients was categorized into three groups: immediate disease progression within the first three months (immediate PD, N=9), partial response or stable disease (PR/SD, N=19), and late disease progression after initial PR/SD (late PD, N=29). Patients whose initial methylation scores were above the cancer cutoff of −0.764 and who were followed up for at least six months were included in the analysis.

In general, the initial methylation scores were similar between patients in PR/SD and PD groups, as shown in Table 10.

TABLE 10
Clinical characteristics of patients with advanced lung cancer in a longitudinal
cohort categorized by their responses to the initial treatment - partial
response/stable disease (PR/SD) and progressive disease (PD)

| | All | | | | | | |
|---|---|---|---|---|---|---|---|
| | patients | | PR/SD | | PD | | |
| Characteristics | (N = 57) | | (N = 19) | | (N = 38) | | p value |
| Gender, n (%) | | | | | | | 0.255 |
| Male | 24 | (42.1%) | 6 | (31.6%) | 18 | (47.4%) | |
| Female | 33 | (57.9%) | 13 | (68.4%) | 20 | (52.6%) | |
| Age, in years | | | | | | | 0.011 |
| Mean | 62.3 | | 67.5 | | 61.8 | | |
| Range | 41.5-80.7 | | 48.5-79.8 | | 41.5-80.7 | | |
| Smoking, n (%) | | | | | | | 0.485 |
| Never | 40 | (70.2%) | 15 | (78.9%) | 25 | (65.8%) | |
| Active | 11 | (19.3%) | 2 | (10.5%) | 9 | (23.7%) | |
| Former | 6 | (10.5%) | 2 | (10.5%) | 4 | (10.5%) | |
| Stage, n (%) | | | | | | | 0.010 |
| Stage IIIB | 4 | (7.0%) | 4 | (21.1%) | 0 | (0%) | |
| Stage IV | 53 | (93.0%) | 15 | (78.9%) | 38 | (100%) | |
| EGFR mutation, n (%) | 48 | (84.2%) | 17 | (89.5%) | 31 | (81.6%) | 0.703 |
| cfDNA size, in base pairs | | | | | | | 0.003 |
| Median | 221 | | 232 | | 217 | | |
| Range | 105-275 | | 188-238 | | 105-275 | | |
| cfDNA conc., ng/mL | | | | | | | 0.411 |
| Median | 35.1 | | 35.1 | | 35.1 | | |
| Range | 14.4-201.6 | | 21.6-160.2 | | 14.4-201.6 | | |
| Initial methylation score | | | | | | | 0.748 |

| | | | |
|---|---|---|---|
| Median | 2.1 | 2.1 | 2.2 |
| Range | −1.6-54.7 | −1.6-54.7 | −0.9-47.0 |

A significant reduction in methylation risk scores following initial systemic treatments in the PR/SD group was observed. On the other hand, FIG. 14B shows the methylation scores were initially decreased but elevated when patients experienced disease progression (PD). The generalized estimating equations (GEE) approach that adjusts for repeated measurements from each visit was applied. In FIG. 14C, a trend of a higher estimated marginal mean was found in the PD group (5.5 vs. 2.9, marginal mean difference, 2.63; 95% C.I., −0.6-5.9).

Table 11 shows that the GEE model has an odds ratio of 0.251 (95% C.I. 0.075-0.84) in the non-PD group, indicating smaller dynamic changes in methylation scores during the treatment when considered the effect of repeated visits (interaction term of PD and visit). The data suggested that methylation risk scores have a great potential for serial disease monitoring.

TABLE 11
Generalized estimating equations (GEE) analysis of the association between
methylation score, disease status and treatment time in a longitudinal
cohort of patients with advanced non-small cell lung cancer

| | | | 95% | 95% | |
|---|---|---|---|---|---|
| Parameter | β | Exp(B) | Lower C.I. | Upper C.I. | p |
| Intercept | 4.262 | 70.957 | 2.107 | 2389.953 | 0.018 |
| Without PD | 2.293 | 9.905 | 0.043 | 2264.133 | 0.408 |
| With PD | reference | 1 | | | |
| Visit | 0.344 | 1.41 | 0.539 | 3.693 | 0.484 |
| Without PD * Visit | −1.38 | 0.251 | 0.075 | 0.84 | 0.025 |
| PD* Visit | reference | 1 | | | |

C.I.: Confidence interval

### Application of Methylation Risk Scores in Disease Monitoring of EGFR Wild-type and Mutated Lung Cancers

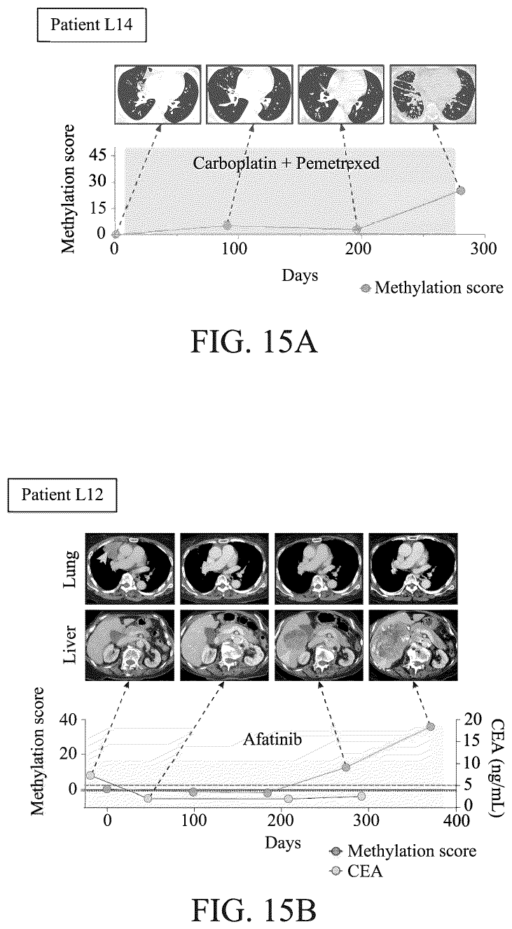

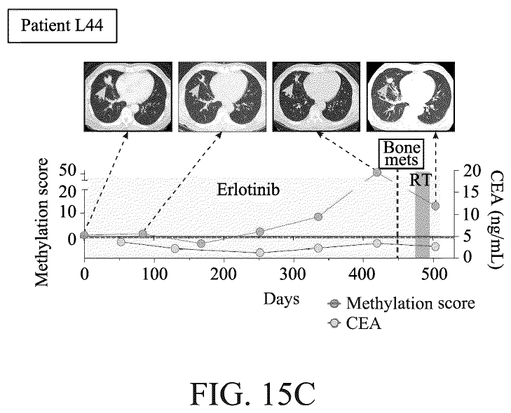

To exemplify the clinical utility of methylation risk scores for disease monitoring in lung cancer patients with or without specific driver mutations who received different therapies, the dynamic changes of methylation risk scores were examined in individual patients as shown in FIGS. 15A to 15C.

Patient L14 was a 56-year-old patient presenting with stage 4 lung adenocarcinoma negative for major driver mutations. The methylation scores did not show significant changes under carboplatin and pemetrexed treatment for about six months, which was correlated with stable disease. At approximately nine months, the methylation score skyrocketed, and clinical progression was evident.

Patient L12 was a 72-year-old patient presenting with EGFR mutated lung adenocarcinoma receiving afatinib treatment. The patient responded to the treatment initially and the methylation scores decreased. However, the score increased again at approximately 270 days despite the shrinkage of the primary lung tumor on chest tomography. Instead, an enlarging metastatic lesion in the liver that correlated with the elevation of methylation scores was discovered. Conversely, there were no changes in the serum levels of carcinoembryonic antigen (CEA).

Patient L44 was a 69-year-old patient with stage 4 EGFR-mutated lung adenocarcinoma receiving erlotinib treatment. The patient's methylation risk scores gradually decreased after therapy. Nevertheless, increased methylation scores were observed at nine months despite the shrinkage of the primary lung tumor on chest tomography. Another six months later, new bone metastases at the lumbar spine and pelvis were finally noted, indicating that elevation of methylation scores preceded evident radiographic and clinical progression. Remarkably, the methylation score declined rapidly after local palliative radiotherapy (RT). However, the CEA levels remained low throughout the entire course.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. A method to characterize non-small cell lung cancer in a subject in need thereof, comprising detecting a methylation level of at least one gene selected from the group consisting of KCNS2, HOXA9, SCT, BARHL2, and any combination thereof in a biological sample from the subject, wherein the biological sample contains circulating free DNA.

2. The method of claim 1, wherein the at least one gene is KCNS2.

3. The method of claim 1, wherein the methylation level is detected by bisulfite sequencing, array or bead hybridization, quantitative real-time PCR, methylation-sensitive endonuclease digestion followed by sequencing, PCR and sequencing, methylation-specific PCR and pyrosequencing .

4. The method of claim 1, wherein the methylation level is detected by at least one primer pair and at least one probe, and wherein:
the at least one primer pair has at least 80% sequence identity to SEQ ID NOs: 1 and 2, respectively, and the at least one probe having at least 80% sequence identity to SEQ ID NO: 3;
the at least one primer pair has at least 80% sequence identity to SEQ ID NOs: 4 and 5, respectively, and the at least one probe having at least 80% sequence identity to SEQ ID NO: 6;
the at least one primer pair has at least 80% sequence identity to SEQ ID NOs: 7 and 8, respectively, and the at least one probe having at least 80% sequence identity to SEQ ID NO: 9;
the at least one primer pair has at least 80% sequence identity to SEQ ID NOs: 10 and 11, respectively, and the at least one probe having at least 80% sequence identity to SEQ ID NO: 12; or any combination thereof.

5. The method of claim 1, further comprising calculating a methylation risk score based on the methylation level of the at least one gene.

6. The method of claim 5, wherein the methylation level of the at least one gene is given different weights for calculating the methylation risk score.

7. The method of claim 6, wherein the calculation of the methylation risk score is further based on at least one of age, gender, active smoking status and former smoking status.

8. The method of claim 7, wherein the at least one of age, gender, active smoking status and former smoking status is given different weights for calculating the methylation risk score.

9. The method of claim 1, wherein the biological sample is a body fluid selected from the group consisting of blood, sputum, pleural fluid, cerebrospinal fluid and any combination thereof.

10. The method of claim 1, wherein the subject carries a driver mutation, a passenger mutation, or a combination thereof.

11. The method of claim 1, further comprising providing a diagnosis of the non-small cell lung cancer.

12. The method of claim 11, wherein the diagnosis is provided at an early stage or a late stage of the non-small cell lung cancer.

13. A kit for characterizing non-small cell lung cancer, the kit comprising a primer pair and a probe for detecting a methylation level of at least one gene, wherein:
the primer pair has at least 80% sequence identity to SEQ ID NOs: 1 and 2, respectively, and the probe has at least 80% sequence identity to SEQ ID NO: 3;
the primer pair has at least 80% sequence identity to SEQ ID NOs: 4 and 5, respectively, and the probe has at least 80% sequence identity to SEQ ID NO: 6;
the primer pair has at least 80% sequence identity to SEQ ID NOs: 7 and 8, respectively, and the probe has at least 80% sequence identity to SEQ ID NO: 9;
the primer pair has at least 80% sequence identity to SEQ ID NOs: 10 and 11, respectively, and the probe has at least 80% sequence identity to SEQ ID NO: 12; or any combination thereof.

14. A method of evaluating a therapy of non-small cell lung cancer, comprising:
obtaining a biological sample comprising circulating free DNA from a subject who had received the therapy of non-small cell lung cancer;
detecting a methylation level of at least one gene selected from the group consisting of KCNS2, HOXA9, SCT, BARHL2, or any combination thereof;
calculating a methylation risk score based on the methylation level of the at least one gene,
wherein a change of the methylation risk score is indicative of efficacy of the therapy of non-small cell lung cancer.

15. The method of claim 14, wherein the at least one gene is KCNS2.

16. The method of claim 15, wherein an increase of the methylation risk score is indicative of disease progression.

17. The method of claim 16, wherein the disease progression comprises increase in tumor size or metastasis.

18. The method of claim 14, wherein the therapy of non-small cell lung cancer is surgery, radiation therapy, chemotherapy, targeted therapy, immunotherapy or any combination thereof.
